(19) United States
(12) Patent Application Publication
(10) Pub. No.: US 2020/0375480 A1
(43) Pub. Date: Dec. 3, 2020

(54) Non-Invasive Cardiovascular Risk Assessment Using Heart Rate Variability Fragmentation

(71) Applicant: BETH ISRAEL DEACONESS MEDICAL CENTER, INC., Boston, MA (US)

(72) Inventors: Madalena D. COSTA, Brookline, MA (US); Ary L. GOLDBERGER, Newton Center, MA (US)

(73) Assignee: BETH ISRAEL DEACONESS MEDICAL CENTER, INC., Boston, MA (US)

(21) Appl. No.: 16/497,331

(22) PCT Filed: Mar. 23, 2018
(86) PCT No.: PCT/US2018/024107

§ 371 Date: Sep. 24, 2019

Related U.S. Application Data
(60) Provisional application No. 62/476,392, filed on Mar. 24, 2017.

(51) Int. Cl. A61B 5/024; A61B 5/0245; A61B 5/0468; A61B 5/00

(52) U.S. Cl. CPC A61B 5/02405; A61B 5/7275; A61B 5/0468; A61B 5/0245

(57) ABSTRACT

Disclosed herein are example methods and systems for non-invasive cardiovascular risk assessment using heart rate variability fragmentation. A first set of electrocardiogram (ECG) signals may be received from a subject. Data from the first set of ECG signals may be analyzed to identify sign changes in heart rate acceleration in the first set of ECG signals. Based on the identified sign changes in heart rate acceleration, a degree of fragmentation in the first set of ECG signals may be determined. Afterwards, cardiovascular risk of the subject may be assessed based on the degree of fragmentation.

### STATEMENT REGARDING FEDERALLY-SPONSORED RESEARCH AND DEVELOPMENT

This invention was made with U.S. Government support under grants Grant Nos. GM104987 and HL114473 awarded by National Institutes of Health (NIH). The U.S. Government has certain rights in the invention.

### BACKGROUND OF THE INVENTION

### Field of the Invention

Embodiments herein relate to systems and methods for assessing cardiovascular risk by fragmentation of heartbeat variability.

### Background Art

Heart rate variability (HRV) is the physiological phenomenon of variation in the time interval between heartbeats for an individual. Short-term HRV is most commonly attributed to physiologic vagal tone modulation, and the degree of short-term variability of normal-to-normal (NN) sinus beats may be used as a dynamic biomarker of cardiac vagal tone modulation.

However, with aging and cardiovascular disease, the emergence of high short-term HRV, consistent with the breakdown of the neuroautonomic-electrophysiologic control system, may confound traditional HRV analysis. For example, parasympathetic regulation of sinus rhythm may decrease with aging and organic heart disease, yet the amount of short-term variability may increase for some subjects in such high risk groups.

Ultimately, the presence of abnormal variants of sinus rhythm may limit the utility of traditional HRV analysis because an increase in the overall amount of short-term variability might not be solely attributed to fluctuations in vagal tone. Therefore, a need exists for new systems, methods, and techniques for analyzing short-term HRV and distinguishing differences in the structure of fluctuations between physiologic and anomalous variability.

### SUMMARY OF THE INVENTION

Example methods and systems are described herein for non-invasive cardiovascular risk assessment using a heart rate variability fragmentation approach. The degree of heartbeat fragmentation may indicate a breakdown of fluency in heartbeats resulting from aging, disease, and/or pathological conditions. In some embodiments, mathematical analysis of a change in sign of a heartbeat acceleration/deceleration signal is performed in order to measure fragmentation of heartbeats. The heartbeat comprises a speed/velocity signal, and an acceleration/deceleration represents a change in the heart rate signal. In some embodiments, the degree of heartbeat fragmentation may indicate a breakdown of fluency in heartbeats resulting from aging, disease, and/or a pathological condition.

In an embodiment, a method of assessing cardiovascular risk of a subject may include receiving a first set of electrocardiogram (ECG) signals of the subject, analyzing data from the first set of ECG signals to identify sign changes in heart rate acceleration in the first set of ECG signals, determining a degree of fragmentation in the first set of ECG signals based on the identified sign changes in heart rate acceleration, and assessing cardiovascular risk of the subject based on the degree of fragmentation. Analyzing data from the first set of ECG signals may further comprise deriving a time series of normal-to-normal (NN) interbeat intervals from each ECG signal and computing a set of fragmentation indices from the time series derived from each ECG signal. The set of fragmentation indices may be applied to the data from the first set of ECG signals.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the present invention and to enable a person skilled in the relevant art(s) to make and use the present invention.

FIGS. 1A-1D illustrate examples of respiratory sinus arrhythmia and anomalous sinus rhythm, according to an embodiment of the present disclosure.

FIG. 2 illustrates a table of Spearman rank and standardized Pearson product-moment coefficients for the relationships between traditional short-term HRV, nonlinear, and fragmentation indices with cross-sectional age for the group of healthy subjects, according to an embodiment of the present disclosure.

FIG. 3 illustrates scatter plots of the traditional heart rate variability (rMSSD, pNN50 and HF), nonlinear (α₁ and SampEn (sample entropy)) and fragmentation (PIP, IALS, PSS and PAS) indices versus the participants' age for the group of healthy subjects and patients with coronary artery disease (CAD), derived from the analysis of the full (˜24-hour) period, according to an embodiment of the present disclosure.

FIG. 4 illustrates a table of measured values of heart rate variability in healthy subjects and measured values of heart rate variability of subjects with coronary artery disease (CAD), according to an embodiment of the present disclosure.

FIG. 5 illustrates a table of values obtained from logistic regression analysis and area under the ROC curve for unadjusted models of CAD, according to an embodiment of the present disclosure.

FIG. 6 illustrates normalized histograms of the traditional heart rate variability (rMSSD, pNN50 and HF), nonlinear (α₁ and SampEn) and fragmentation (PIP, IALS, PSS and PAS) indices for the groups of healthy subjects (blue) and patients with coronary artery disease (red), for the 24-hour period, according to an embodiment of the present disclosure.

FIG. 7 illustrates a table of values obtained from logistic regression analysis and AUC for models of CAD adjusted for age and sex, according to an embodiment of the present disclosure.

FIG. 8 illustrates scatter plots of the traditional heart rate variability (rMSSD, pNN50 and HF), nonlinear (α₁ and SampEn) and fragmentation (PIP, IALS, PSS and PAS) indices versus mean heart rate (in beats per minute, bpm) for the group of healthy subjects (blue dots) and those with coronary artery disease (CAD, red circles), derived from the analysis of 24-hour NN interval time series, according to an embodiment of the present disclosure.

FIG. 9 illustrates examples of respiratory sinus arrhythmia and anomalous (fragmented) sinus rhythm, according to an embodiment of the present disclosure. Electrocardiograms (Holter lead) from a healthy subject (first row) and a patient with coronary artery disease (CAD) (second row), both from the present study. Normal-to-normal (NN) sinus interval time series from the healthy subject (third row, left) and the patient with CAD (third row, right). The fluctuation patterns of the former time series are characteristic of phasic (respiratory) sinus arrhythmia, while that of the latter are indicative of an abnormal, non-phasic sinus arrhythmia. See Costa, M. D., Davis, R. B., and Goldberger, A. L. (2017). Heart rate fragmentation: a new approach to the analysis of cardiac interbeat interval dynamics. Front. Physiol. 8:255 (herein “Costa I 2017”). Positive and negative changes in the value of the NN intervals, corresponding to heart rate decelerations and accelerations were mapped to symbols “−1” and “1,” respectively. Symbol “0” is used to represent intervals in which heart rate did not change. To assist in visual comparisons, pale gray backgrounds are used for data from the healthy subject and light red for data from the patient with CAD, respectively. The symbolic mapping of the differences between consecutive NN intervals for the ECG of the healthy subject (first 16 intervals) along with the first four words that were derived from this sequence are shown on the bottom left. The first word “−1−111” contains one hard inflection point. It belongs to the group W₁ and, more specifically, to the subgroup W₁^{H}. The following three words, “−1110,” “110−1,” and “10−1−1” contain two inflection points. Therefore, they belong to group W₂. However, the first word (“−1110”) belongs to the subgroup W₂^{M} since it contains one hard and one soft inflection point; the second (“110−1”) and the third (“10−1−1”) words belong to the subgroup W₂^{S} since they present two soft inflection points. The panels on the bottom right show the percentage of words in each group for the healthy subject (left) and patient with CAD (right). Note a substantially higher percentage of fragmented words for the patient with CAD than for the healthy subject. The abbreviation “a.u.” stands for arbitrary units.

FIG. 10 illustrates a schematic diagram of 81 different words of length 4 with an alphabet of 3 symbols, in which the symbols “/”, “\”, and “−” represent heart rate acceleration, deceleration and no change, respectively. Words were grouped by the number and type of inflection points. The labels, 0-80, shown in parentheses, are the decimal value of the ternary representation of each pattern using the symbols “2” if ΔNNᵢ<0, “1” if ΔNNᵢ>0 and “0” if ΔNNᵢ=0. For example, the label for the word comprising 4 consecutive accelerations, i.e., the word 2222, is 80 (=2×3³+2×3²+2×3¹+2×3⁰). Abbreviations: W, word subgroup. The subscript and superscript of W indicate, respectively, the number and the type of inflection points, hard (H), soft (S) or a combination of hard and soft (M, mixed) that the words in that subgroup contain.

FIG. 11 illustrates a table of slope and [95% confidence intervals] of the association between each outcome measure and the participants' age for the group of healthy subjects and those with CAD, for the 24-h and putative awake and sleep periods. CAD, coronary artery disease; PIP, percentage of inflection points; Wⱼ, 0≤j≤3, group of words containing j inflection points; superscripts: H, hard inflection points; S, soft inflection points; M, mixed inflection points, i.e., a combination of hard and soft inflection points. Word groups for which the type of inflection point is not specified comprise words with all types of inflection points. The percentages of words in the groups Wⱼ^{H}* and Wⱼ^{S}* were calculated over the total number of NN words with only hard and only soft inflection points, respectively. The percentages of words in the other groups were calculated over the total number of NN words. Slope values marked with the symbol † are significantly different in the two sample populations.

FIGS. 12A-12C illustrate graphs depicting the relationship between the percentage of words with no inflection points (W₀), one (W₁), two (W₂) and three (W₃) inflection points and the participants' age for the healthy subjects (blue) and those with coronary artery disease (CAD, red) during the 24-h (FIG. 12A), putative awake (FIG. 12B) and putative sleep (FIG. 12C) periods. Symbols and lines represent, respectively, word percentages for each subject and the regression lines derived from linear regression analyses controlled for the average NN interval. In each plot, the rates of change of the outcome variables per year of age for the healthy subjects and the patients with CAD are indicated in blue and red, respectively.

FIG. 13 illustrates a table showing measures of heart rate fragmentation/fluency in healthy subjects and those with coronary artery disease. Values are reported as median, 25th-75th percentiles. CAD, coronary artery disease; PIP, percentage of inflection points; Wⱼ, 0≤j≤3, group of words containing j inflection points; superscripts: H, hard inflection points; S, soft inflection points; M, mixed inflection points, i.e., a combination of hard and soft inflection points. Word groups for which the type of inflection point is not specified comprise words with all types of inflection points. The percentages of words in the groups Wⱼ^{H}* and Wⱼ^{S}* were calculated over the total number of NN words with only hard and only soft inflection points, respectively. The percentages of words in the other groups were calculated over the total number of NN words.

FIG. 14 illustrates a table showing logistic regression analysis and area under the ROC curve for unadjusted models of CAD. Values presented are normalized odds ratio (ORₙ), 95% confidence intervals (95% CI) and area under the receiver operating characteristic curve (AUC). CAD, coronary artery disease; PIP, percentage of inflection points; Wⱼ, 0≤j≤3, group of words containing j inflection points; superscripts: H, hard inflection points; S, soft inflection points; M, mixed inflection points, i.e., a combination of hard and soft inflection points. Word groups for which the type of inflection point is not specified comprise words with all types of inflection points. The percentage of word groups without “*” was calculated over the total number of NN words. The percentages of words in the groups Wⱼ^{H}* and Wⱼ^{S}* were calculated over the total number of NN words with only hard and only soft inflection points, respectively. The percentages of words in the other groups were calculated over the total number of NN words.

FIG. 15 illustrates a table showing logistic regression analysis and AUC for models of CAD adjusted for age and sex. The analysis was performed using raw measures. Values presented are the normalized odds ratio (ORn) and the 95% confidence intervals (95% CI) for the variables listed in the header column, in models adjusted for age and sex; the area under the receiver operating characteristic curve (AUC) and the p value for the likelihood-ratio test of the null hypothesis that the addition of the HRV measure does not improve the fit of the model with age and sex alone. CAD, coronary artery disease; PIP, percentage of inflection points; Wⱼ, 0≤j≤3, group of words containing j inflection points; superscripts: H, hard inflection points; S, soft inflection points; M, mixed inflection points, i.e., a combination of hard and soft inflection points. Word groups for which the type of inflection point is not specified comprise words with all types of inflection points. The percentages of words in the groups Wⱼ^{H}* and Wⱼ^{S}* were calculated over the total number of NN words with only hard and only soft inflection points, respectively. The percentages of words in the other groups were calculated over the total number of NN words.

FIG. 16 illustrates a table showing logistic regression analysis and AUC for models of CAD adjusted for age, sex, and the average value of the NN intervals. The analysis was performed using raw measures. Values presented are the normalized odds ratio (ORₙ) and the 95% confidence intervals (95% CI) for the variables listed in the header column, in models adjusted for age, sex and the average value of the NN intervals (AVNN); the area under the receiver operating characteristic curve (AUC) and the p value for the likelihood-ratio test of the null hypothesis that the addition of the HRV measure does not improve the fit of the model with age and sex alone. CAD, coronary artery disease; PIP, percentage of inflection points; Wⱼ, 0≤j≤3, group of words containing j inflection points; superscripts: H, hard inflection points; S, soft inflection points; M, mixed inflection points, i.e., a combination of hard and soft inflection points. Word groups for which the type of inflection point is not specified comprise words with all types of inflection points. The percentages of words in the groups Wⱼ^{H}* and Wⱼ^{S}* were calculated over the total number of NN words with only hard and only soft inflection points, respectively. The percentages of words in the other groups were calculated over the total number of NN words.

FIG. 17 illustrates a table with characteristics of Multi-Ethnic Study of

Atherosclerosis (MESA) participants without and with a cardiovascular event (CVE) during follow-up. Values presented are the population mean and SD for continuous variables and the number of participants and its percentage for categorical variables. Abbreviations: BMI, body mass index; HR, heart rate; BP, blood pressure; HDL, high density lipoprotein; CVE, cardiovascular event; SD, standard deviation.

FIG. 18 illustrates a table showing the association of fragmentation and traditional HRV indices with incident CVEs in unadjusted and adjusted models for standard risk factors. In particular, FIG. 18 shows Models 1 and 2; Model 1: unadjusted. Model 2: adjusted for the traditional risk factors: age, sex, systolic blood pressure, total cholesterol, HDL cholesterol, current smoking status, hypertension medication, diabetes and lipid lowering medication. Values presented are standardized hazard ratios (HRs), 95% confidence intervals (95% CI), Harrell's C statistic (C-index) and the p-value for the likelihood ratio test of the null hypothesis that the addition of a dynamical measure (fragmentation or HRV metric) to a model with the traditional risk factors did not improve the fit of data. The numbers of participants/events in the analyses of the models 1 and 2 were 1771/72 and 1702/71, respectively. Abbreviations: HRV, heart rate variability; CVE, cardiovascular event; HDL, high density lipoprotein; PIP, percentage of inflection points; W₀, W₁, W₂, W₃, percentage of words with 0, 1, 2 and 3 inflection points; AVNN, average value of the NN intervals; SDNNIDX, mean of the standard deviations of NN intervals in all 5-minute segments; rMSSD, root mean square of the successive differences; pNN50, percentage of differences between successive NN intervals above 50 ms; HF, high frequency spectral power; LF/HF, ratio of low to high frequency power.

FIG. 19 illustrates a table showing the association of fragmentation and traditional

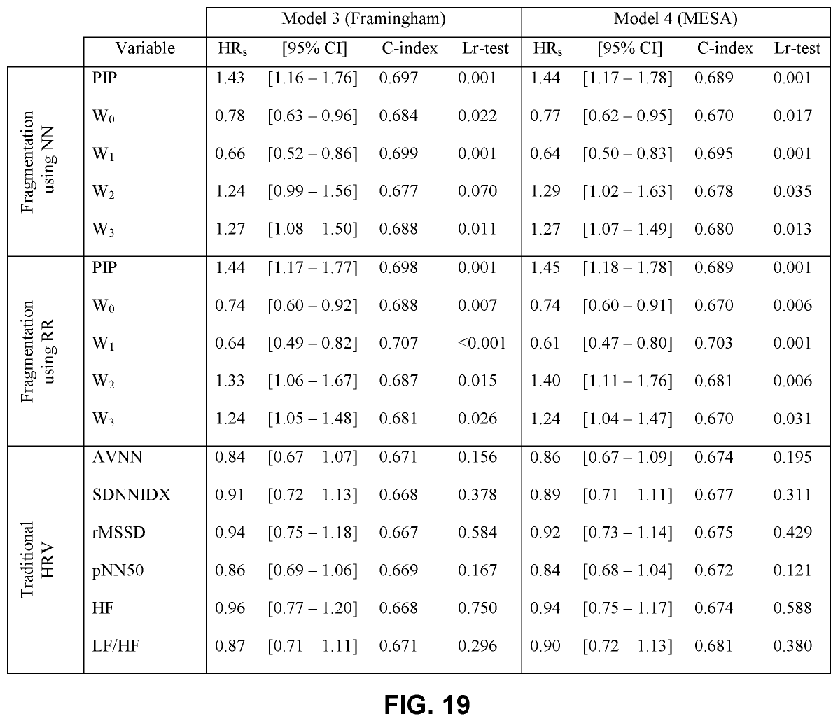

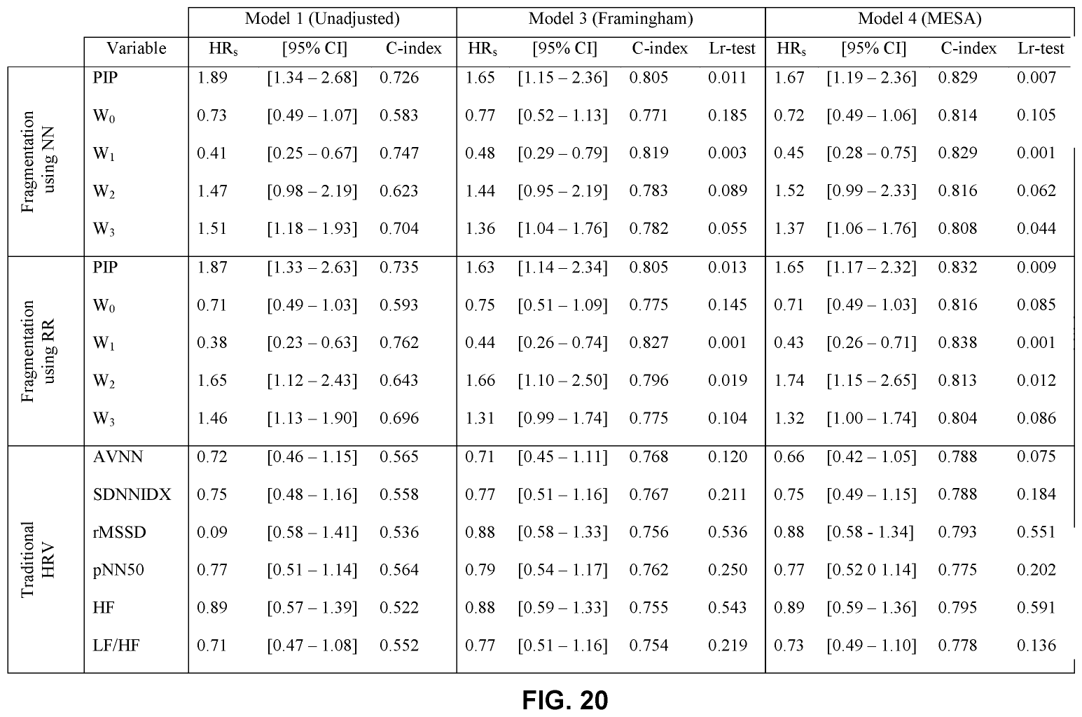

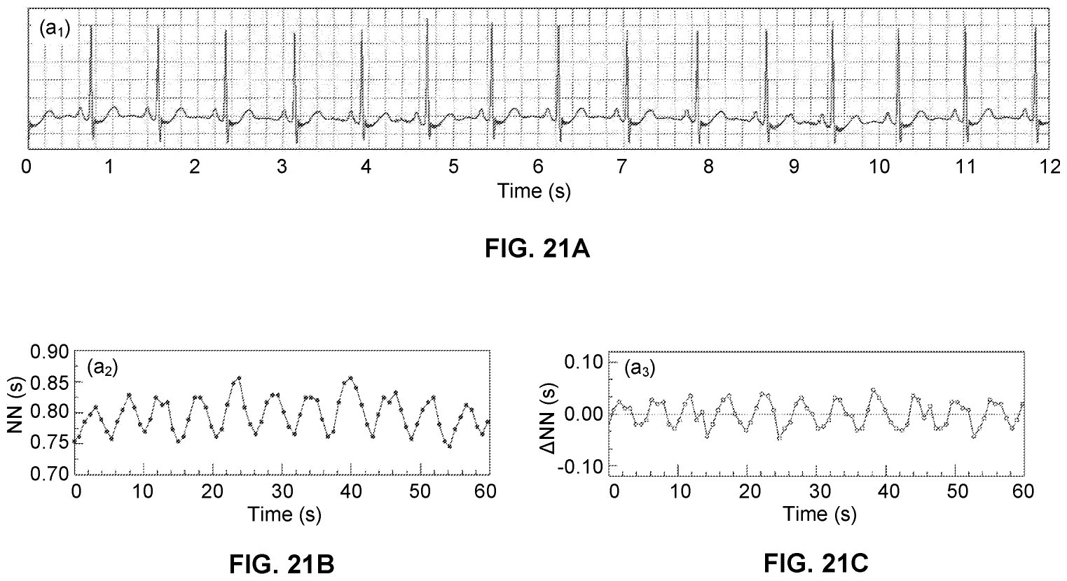

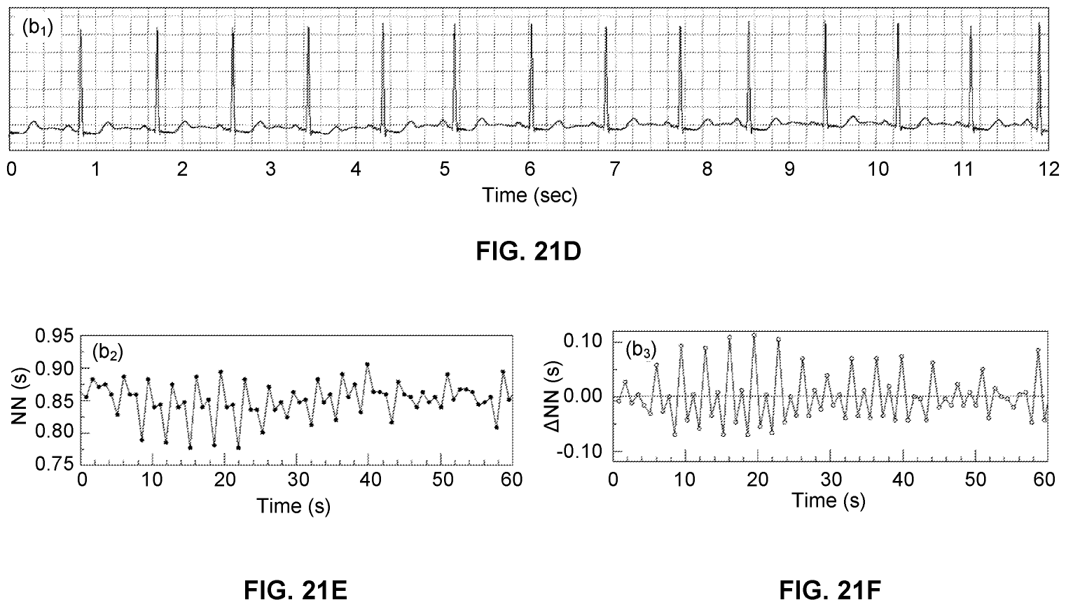

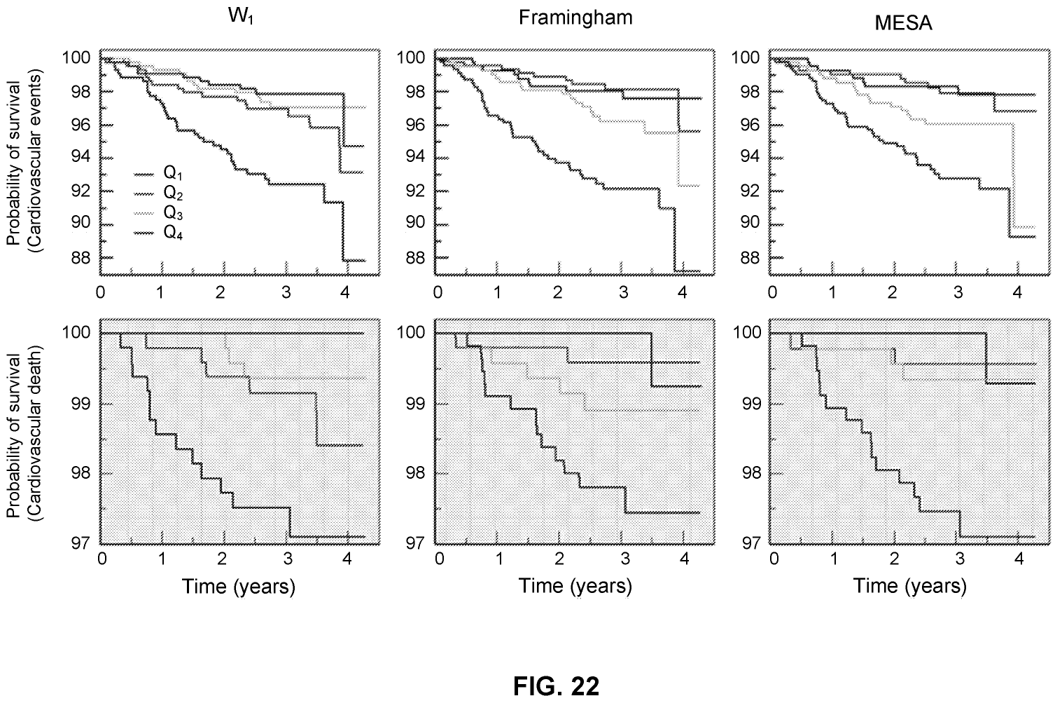

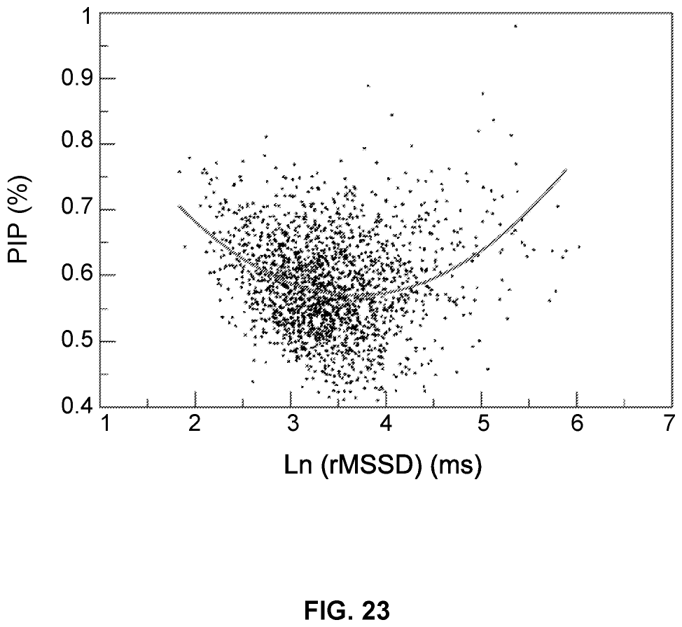

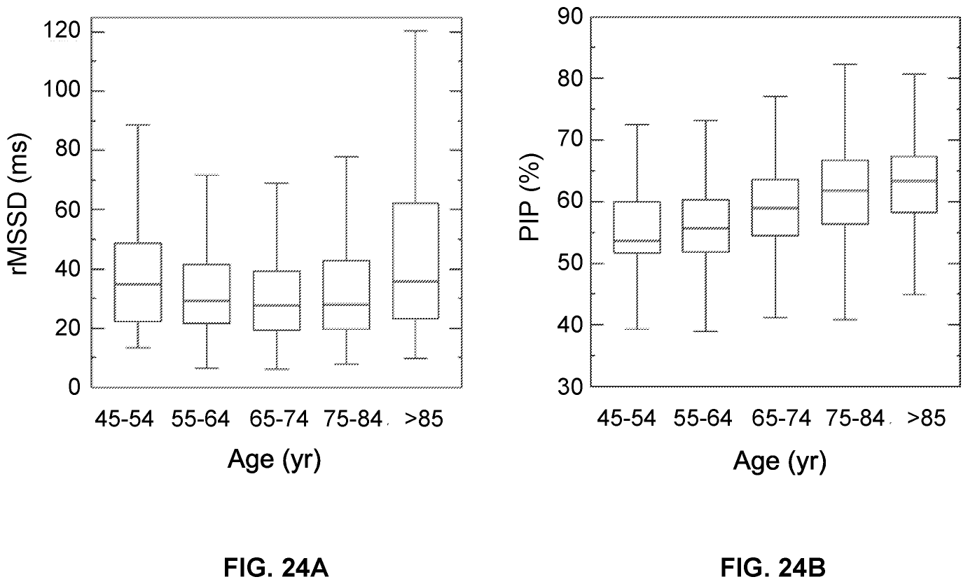

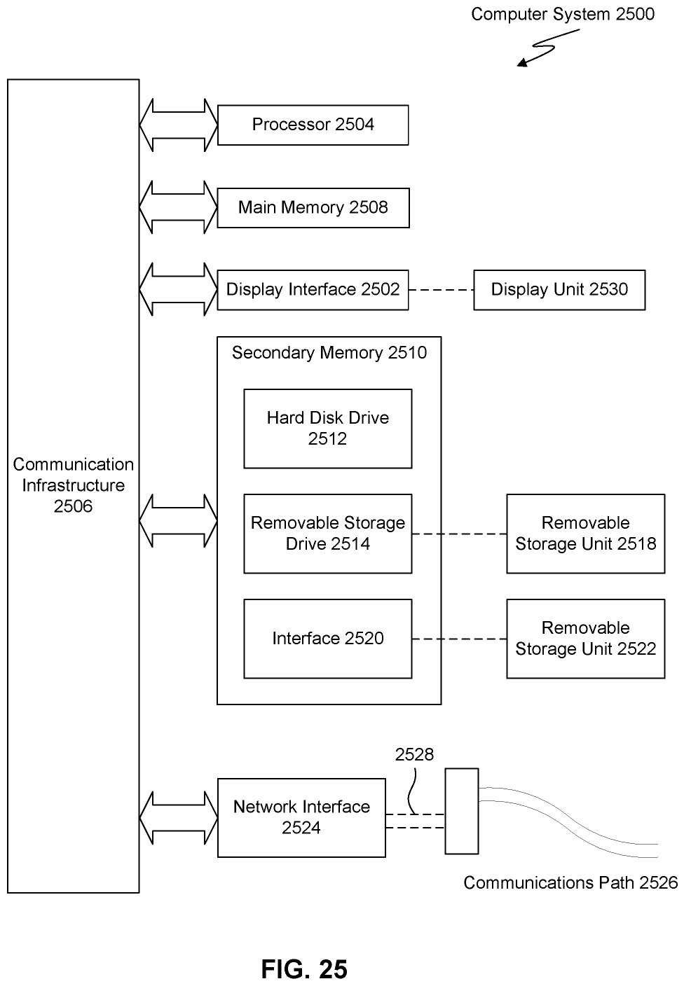

HRV indices with incident CVEs in models adjusted for the Framingham and MESA CV risk indices. In particular, FIG. 19 shows Models 3 and 4; Model 3: adjusted for the Framingham CV risk index. See R. B. D'Agostino, R. S. Vasan, M. J. Pencina, P. A. Wolf, M. Cobain, J. M. Massaro, and W. B. Kannel. General cardiovascular risk profile for use in primary care: the Framingham Heart Study. Circulation, 117(6):743-753, 2008 (herein “D'Agostino 2008”). Model 4: adjusted for the MESA CV risk index. Values presented are standardized hazard ratios (HRs), 95% confidence intervals (95% CI), Harrell's C statistics (C-index) and the p-value for the likelihood ratio test of the null hypothesis that the addition of an HRV metric to a model with one of three risk indices did not improve the fit of the data. The numbers of participants/events in the analyses of the models 3, 4 and 5 were 1767/72, 1672/71 and 1305/55 respectively. Abbreviations: HRV, heart rate variability; CVE, cardiovascular event; CV, cardiovascular; PIP, percentage of inflection points; W₀, W₁, W₂, W₃, percentage of words with 0, 1, 2 and 3 inflection points; AVNN, average value of the normal-to-normal sinus (NN) intervals; SDNNIDX, mean of the standard deviations of NN intervals in all 5-minute segments; rMSSD, root mean square of the successive differences; pNN50, percentage of differences between successive NN intervals above 50 ms; HF, high frequency spectral power; LF/HF, ratio of low to high frequency power.

FIG. 20 illustrates a table showing the association of fragmentation and traditional HRV indices with CV death in models adjusted for the Framingham and MESA CV risk indices. In particular, FIG. 20 shows Models 1-3; Model 1: unadjusted. Model 2: adjusted for the Framingham CV risk index per D′Agostino 2008. Model 3: adjusted for the MESA CV risk index. The numbers of participants in the analyses of the models 1, 2 and 3 were 1963, 1958 and 1856, respectively. The number of participants/events in models 1, 2 and 3 was 1963/21, 1958/21 and 1856/21, respectively. Values presented are standardized hazard ratios (HRs), 95% confidence intervals (95% CI), Harrell's C statistics (C-index) and the p-value for the likelihood ratio test of the null hypothesis that the addition of a dynamical measure (fragmentation or HRV metric) to a model with one of the risk indices did not improve the fit of the data. Abbreviations: HRV, heart rate variability; CV, cardiovascular; PIP, percentage of inflection points; W₀, W₁, W₂, W₃, percentage of words with 0, 1, 2 and 3 inflection points; AVNN, average value of the normal-to-normal sinus (NN) intervals; SDNNIDX, mean of the standard deviations of NN intervals in all 5-minute segments; rMSSD, root mean square of the successive differences; pNN50, percentage of differences between successive NN intervals above 50 ms; HF, high frequency spectral power; LF/HF, ratio of low to high frequency power.

FIGS. 21A-21F illustrate examples of twelve-second electrocardiographic (ECG) recordings (shown in FIGS. 21A and 21D), normal-to-normal (NN) sinus interval time series (shown in FIGS. 21B and 21E), and ANN (increment) time series (shown in FIGS. 21C and 21F) from subjects with respiratory sinus arrhythmia and fragmented sinus rhythm. In particular, FIGS. 21A-21C show examples of respiratory sinus arrhythmia for Subject A, a 74 year-old African-American male without incident cardiovascular events, and FIGS. 21D-21F show examples of fragmented sinus rhythm for Subject B, a 77 year-old Caucasian female with incident events (transient ischemic attack, percutaneous coronary angioplasty and coronary revascularization) 382 days after the polysomnographic study. Note that the former shows a more “fluent,” less fragmented interbeat interval pattern than the latter. However, the ECG rhythm strips are both clinically consistent with “normal sinus rhythm.”

FIG. 22 illustrates example Kaplan-Meier survival curves of analyses of incident CVEs (top panels) and CV mortality(bottom panels), showing the percentage of symbolic words with one inflection point derived (W₁) from RR interval time series (left panels), the Framingham (middle panels) and MESA (right panels) CV risk indices.

FIG. 23 illustrates an example scatterplot of the natural logarithm of rMSSD and PIP. The solid line is described by PIP=−0:333*ln(rMSSD)²+0:046*[ln(rMSSD)]²+1.175. The 95% CI for ln(rMSSD), [ln(rMSSD)]² and the constant term were [−0.375, −0.291], [0.040, 0.052] and [1.101, 1.249], respectively. Abbreviations: PIP, percentage of inflection points; rMSSD, root mean square of the successive differences; CI, confidence interval.

FIGS. 24A and 24B illustrate example Tukey boxplots of ln(rMSSD) and PIP for participants in successive age groups. Abbreviations: PIP, percentage of inflection points; rMSSD, root mean square of the successive differences.

FIG. 25 illustrates an example computer system useful for implementing portions of the present invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

This specification discloses one or more embodiments that incorporate the features of this invention. The disclosed embodiment(s) merely exemplify the present invention. The scope of the present invention is not limited to the disclosed embodiment(s).

The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

### LIST OF ABBREVIATIONS USED HEREIN

α₁—detrended fluctuation analysis short-term exponent
a.u.—Arbitrary units
AUC—Area under the receiver operating characteristic curve
AVNN—average value of the NN intervals
BMI—body mass index
BP—blood pressure
CAD—Coronary artery disease
CV—cardiovascular
CVD—cardiovascular disease
CVE—cardiovascular event
DFA—Detrended fluctuation analysis
ECG—Electrocardiogram
F(n)—DFA root-mean-square fluctuation function of the integrated and detrended data, computed using windows of length n
HDL—high density lipoprotein
HF—High frequency spectral power (e.g., total spectral power of all NN intervals between 0.15 and 0.4 Hz)
HR—heart rate
HRF—heart rate fragmentation
HRV—heart rate variability
IALS—Inverse of the average length of the acceleration/deceleration segments.
IDEAL—Intercity Digital Electrocardiogram Alliance
LF/HF—ratio of low to high frequency power
MESA—Multi-Ethnic Study of Atherosclerosis
NN—Normal-to-normal (sinus) interbeat interval
ORₙ—Normalized odds ratio
PAS—Percentage of NN intervals in alternation segments
PIP—Percentage of inflection points (e.g., changes in heart acceleration sign)
pNN20—Percentage of differences between adjacent NN intervals that are greater than 20 ms
pNN50—Percentage of differences between adjacent NN intervals that are greater than 50 ms
PSG—polysomnography or polysomnogram
PSS—Percentage of NN intervals in short segments
RR—Cardiac interbeat interval (e.g., R-to-R (interval))
RSA—Respiratory sinus arrhythmia
rMSSD—Root mean square of successive differences (e.g., square root of the mean of the squares of differences between adjacent NN intervals)
SA/SAN—Sino-atrial node
SampEn—Sample entropy
SDNNIDX—mean of the standard deviations of NN intervals in all 5-minute segments
SDSD—Standard deviation of successive differences
SVPB—Supra-ventricular premature beat
THEW—Telemetric and Holter ECG Warehouse.
Wj—segment, termed “word,” of 4 consecutive differences between adjacent interbeat intervals presenting j changes in heart rate acceleration sign.

### INTRODUCTION

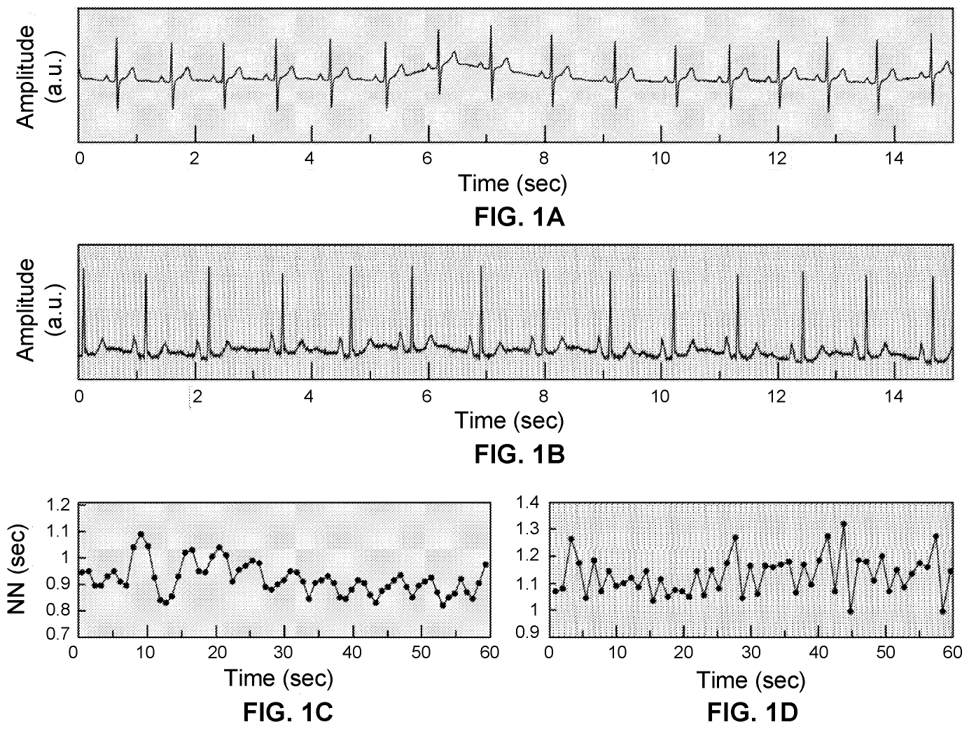

Heart rate variability (HRV) in healthy subjects, particularly over short time scales, is primarily attributable to fluctuations in vagal tone. The most recognizable manifestation of this parasympathetic influence is the oscillatory RR interval pattern (cardiac interbeat interval, e.g., illustrated in FIGS. 1A-1D) termed respiratory sinus arrhythmia (RSA) that results from the coupling between breathing and heart rate. See Heart rate variability: standards of measurement, physiological interpretation 539 and clinical use. Task Force of the European Society of Cardiology and the North American Society of Pacing and Electrophysiology. Circulation 93, 1043-1065, 1996 (herein “Heart rate variability 1996”); Angelone, A. and Coulter Jr, N. A. (1964). Respiratory sinus arrhythmia: a frequency dependent phenomenon. J Appl Physiol 19, 479-482 (herein “Angelone 1964”); Hirsch, J. A. and Bishop, B. (1981). Respiratory sinus arrhythmia in humans: how breathing pattern modulates heart rate. Am J Physiol 241, H620-629 (herein “Hirsch 1981”); Stauss, H. M. (2003). Heart rate variability. Am J Physiol Regul Integr Comp Physiol 285, R927-R931 (herein “Stauss 2003”). However, beat-to-beat changes in the heart rate of healthy subjects not synchronized with respiration are also vagally mediated. See Angelone 1964, Hirsch 1981. Therefore, a central interpretative framework underlying contemporary HRV analyses is one in which the degree of short-term variability of normal-to-normal (NN) sinus beats is used as a dynamical biomarker of cardiac vagal tone modulation. See Heart rate variability 1996, Angelone 1964, and Billman, G. E. (2011). Heart rate variability—a historical perspective. Front Physiol 2, 86 (herein “Billman 2011”).

This topic is of particular importance because parasympathetic regulation of sinus rhythm decreases with aging and organic heart disease. See Heart rate variability 1996; Kuo, T. B., Lin, T., Yang, C. C., Li, C. L., Chen, C. F., and Chou, P. (1999). Effect of aging on gender differences in neural control of heart rate. Am. J. Physiol. 277, H2233-2239 (herein “Kuo 1999”); Thayer, J. F., Yamamoto, S. S., and Brosschot, J. F. (2010). The relationship of autonomic imbalance, heart rate variability and cardiovascular disease risk factors. Int. J Cardiol. 141, 122-131. (herein “Thayer 2010”). However, paradoxically, for some subjects in these high risk groups the amount of short-term variability actually increases (illustrated in FIGS. 1A-1D). For example, FIGS. 1A-1D illustrate examples of respiratory sinus arrhythmia and anomalous sinus rhythm, according to an embodiment of the present disclosure. FIG. 1A illustrates electrocardiograms (Holter lead) from a healthy subject in the present study, whereas FIG. 1B illustrates electrocardiograms from a patient with coronary artery disease (CAD) from the present study. FIG. 1C illustrates normal-to-normal (NN) sinus interval time series from the healthy subject, and FIG. 1D illustrates normal-to-normal (NN) sinus interval time series from the patient with CAD. The fluctuation patterns of the former time series are characteristic of phasic (respiratory) sinus arrhythmia, while that of the latter are indicative of an abnormal non-phasic sinus arrhythmia. To assist in visual comparisons, pale gray backgrounds are used for data from the healthy subject and light red for data from the patient with CAD, respectively. Electrocardiogram (ECG) voltage is given in arbitrary units (a.u.).

An apparent difference in the time series of vagally and non-vagally mediated HRV dynamics is their degree of smoothness, or conversely, their degree of fragmentation. Vagal tone modulation changes the heart rate in a progressive way. For example, with RSA, heart rate gradually increases and decreases with inspiration and expiration, respectively. When the coupling between heart rate and respiration is not as apparent, but the changes in heart rate are still driven by vagal tone modulation, the changes in heart rate are also gradual. In contrast, non-vagally mediated, short-term heart rate variability has a distinct dynamical signature, namely more frequent changes in heart rate acceleration sign (illustrated in FIGS. 1A-1D). In the “extreme” case of sinus alternans, the sign of heart rate acceleration changes every beat. See Binkley, P. F., Eaton, G. M., Nunziata, E., Khot, U., and Cody, R. J. (1995). Heart rate alternans. Ann Intern Med 122, 115-117 (herein “Binkely 1995”); Friedman, B. (1956). Alternation of cycle length in pulsus alternans. Am Heart J 51, 701-712. (herein “Friedman 1956”); Geiger, A. and Goerner, J. (1945). Premature beats of sinus origin: electrocardiographic demonstration of a clinical case. Am Heart J 30, 284-291 (herein “Geiger 1945”); Lewis, T. (1920). The mechanism and graphic registration of the heart beat (PB Hoeber) (herein “Lewis 1920”). The presence of these abnormal variants of sinus rhythm limits the utility of traditional HRV analysis, since an increase in the overall amount of short-term variability can no longer be solely attributed to enhanced vagal tone modulation.

Domitrovich et al. 2002 and Stein 2002 coined the term “erratic sinus rhythm” to refer to prominent but apparently random variations in sinus cadence not attributable to vagal tone modulation and proposed a semi-quantitative approach to help identify them. See Domitrovich, P. P. and Stein, P. K. (2002). A new method to detect erratic sinus rhythm in RR-interval files generated from Holter recordings. Comput Cardiol 26, 665-668 (herein “Domitrovich 2002”); Stein, P. K. (2002). Heart rate variability is confounded by the presence of erratic sinus rhythm. Comput Cardiol 26, 669-672, (herein “Stein 2002”); Stein, P. K., Domitrovich, P. P., Hui, N., Rautaharju, P., and Gottdiener, J. (2005). Sometimes higher heart rate variability is not better heart rate variability: results of graphical and nonlinear analyses. J Cardiovasc Electrophysiol 16, 954-959 (herein “Stein 2005”); Stein, P. K., Le, Q., and Domitrovich, P. P. (2008). Development of more erratic heart rate patterns is associated with mortality post-myocardial infarction. J Electrocardiol 41, 110-115 (herein “Stein 2008”). However, despite their association with increased cardiovascular risk and sick sinus syndrome, erratic sinus rhythm, sinus alternans, and their variants, have received scant clinical attention and the underlying mechanisms remain obscure. See Bergfeldt, L. and Haga, Y. (2003). Power spectral and Poincaré plot characteristics in sinus node dysfunction. J Appl Physiol 94, 2217-2224 (herein “Bergfeldt 2003”).

As illustrated in FIGS. 1A-1D, the distinctions between the different classes of sinus arrhythmia may be difficult or impossible to discern from standard ECG recordings. The graphs of the NN interval time series and other representations of the data, such as Poincare plots and Fourier spectra (not shown) may reveal clear differences in the structure of the fluctuations between physiologic and anomalous variability. However, in many cases, the differences are difficult to identify and especially to quantify.

These considerations led to the development of a novel approach to the analysis of short-term heart rate variability, termed heart rate fragmentation, accompanied by a set of simple-to-implement statistical metrics. A framework for the proposed approach is the concept that adaptive control of the heartbeat, particularly on short time scales, requires a hierarchy of interacting networks comprising neuroautonomic (especially the parasympathetic) and electrophysiologic components (sinus node pacemaker cells and their connections to the atrial syncytium). The integrity of these networks allows for their correlated function, evinced in part by the smoothness (fluency) of the output. At the same time, their functionality provides for sufficiently rapid (short-term or high frequency) responsiveness to physiologic stresses, while protecting against excessive volatility on a beat-to-beat basis.

A corollary concept is that network dysfunction, in general, and of the heart rate control system in particular, is more likely to occur as the components of the network and their physiologic coupling start to break down. This degradative process should lead to increasing degrees of fragmentation. A key aspect of the fragmentation paradigm is that dysfunction or actual breakdown of one or more system components allows for the emergence of high frequency fluctuations that compete with or even exceed the shortest-term modulatory responsiveness of the vagal system. Therefore, a marker of this fragmentation on the surface ECG should be abrupt changes in the sign of heart rate acceleration, which may be periodic (as with classic sinus node alternans) or more random appearing (as with what has been termed “erratic sinus rhythm”). Such markers of fragmentation may be useful as correlates of cardiovascular aging and/or underlying organic heart disease.

Accordingly, a set of fragmentation indices was developed (as described herein) and applied to beat-annotated, well-characterized 24-hour Holter monitor recordings obtained from two very distinct clinical groups: healthy subjects and those with coronary artery disease (CAD). Three different time periods were analyzed: the full day, putative awake and sleep periods. The primary hypotheses were that: 1) heart rate fragmentation would be higher in healthy old subjects than in younger ones for all three time periods; and 2) heartbeat time series from patients with CAD would be more fragmented than those from healthy subjects. The fragmentation indices were also tested to determine whether the fragmentation indices would outperform standard time and frequency domain measures, as well as nonlinear measures of short-term HRV in classifying heart rate time series from healthy subjects versus those from patients with CAD.

### METHODS

### Databases

Two long-term (˜24-hour) ECG ambulatory databases were utilized from the

Intercity Digital Electrocardiogram Alliance (IDEAL) study. The recordings are made available via the University of Rochester Telemetric and Holter ECG Warehouse (THEW) archives (http://thew-project.org/databases.htm).

### 1. Healthy Subjects Database (THEW identification: E-HOL-03-0202-003)

The database comprises 24-hour Holter recordings from 202 ostensibly healthy subjects (102 males). Subjects were not pregnant and had 1) no overt cardiovascular disease or history of cardiovascular disorders; 2) no reported medications, 3) a normal physical examination, 4) a 12-lead ECG showing sinus rhythm with normal waveforms (or a normal echocardiogram and normal ECG exercise testing in the presence of any questionable findings ECG changes). The ECG signals were recorded at a sample frequency of 200 Hz. Automated beat annotations were manually reviewed and adjudicated. The following subjects were excluded: 45 subjects with more than 1% non-sinus beats, 37 younger than 25 years old, ten with body mass index >30 kg/m² and one with <12 hours of data. Overall, data from healthy adult subjects (60 male), age (median, 25^{th}-75^{th} percentiles) 40, 33-49 years, was analyzed.

### 2. Coronary Artery Disease Subjects Database (THEW Identification E-HOL-03-0271-002)

This database comprises 24-hour Holter recordings from 271 patients (223 males).

Subjects had an abnormal coronary angiogram (at least one vessel with luminal narrowing >75%) and either exercise-induced ischemia or a documented previous myocardial infarction. Exclusion criteria included a history of coronary artery bypass surgery or major co-morbidity. Patients were clinically stable and in sinus rhythm at the time of the enrollment. For the analysis, the following subjects were excluded: 11 subjects whose Holter recordings contained ≥20% non-sinus beats and 4 with less than 12 hours of data. Overall, 256 subjects were analyzed: (208 male), age (median, 25^{th}-75^{th} percentiles): 60; 51-67 yrs; left ventricular ejection fraction 56.5, 50-66%.

Putative waking and sleeping periods were estimated as the 144 six consecutive hours of highest and lowest heart rates, respectively. These periods were calculated from the NN interval time series using a six-hour moving average window, shifted 15 minutes at a time.

### HRV Analysis: Heart Rate Fragmentation Indices

From the ECG of each subject, the time series of the NN intervals, {NNᵢ}={t_{Ni}−t_{Ni-1}}, where t_{Ni} represents the time of occurrence of the i^{th} normal sinus beat, and the time series of the differences between consecutive NN intervals (increments), {ΔNNᵢ}={NNᵢ−NNᵢ₋₁}, were derived.

The following four fragmentation indices were then computed from these time series:

(1) The percentage of zero-crossing points in the increment time series, or equivalently, the percentage of inflection points (PIP) in the NN interval time series. (A t_{Ni} represents an inflection point if ΔNNᵢ×ΔNNᵢ₊₁≤0, that is, if t_{N}, is an instant of inversion of heart rate acceleration sign or of change to or from zero).

(2) The inverse of the average length of the acceleration/deceleration segments (IALS). An acceleration, deceleration segment is a sequence of NN intervals between consecutive inflection points for which the difference between two NN intervals is <0 and >0, respectively. The length of a segment is the number of NN intervals in that segment.

(3) The complement of the percentage of NN intervals in acceleration and deceleration segments with three or more NN intervals. This quantity is termed the percentage of short segments (PSS).

(4) The percentage of NN intervals in alternation segments. An alternation segment is a sequence of at least four NN intervals, for which heart rate acceleration changes sign every beat. Such sequences follow an “ABAB” pattern, 168 where “A” and “B” represent increments of opposite sign. This quantity is abbreviated, PAS.

By definition, the more fragmented a time series is, the higher the PIP, IALS, PSS and PAS indices will be. In some cases, PAS quantifies the amount of a particular sub-type of fragmentation (alternation). A time series may be highly fragmented and have a small amount of alternation. However, all time series with large amount of alternation are highly fragmented.

Given that the presence of non-sinus beats will increase fragmentation, segments encompassing non-sinus beats that started and ended at the inflection points preceding and following these non-sinus beats, respectively, were excluded. Finally, to assess the importance of beat annotation on fragmentation analyses, the full RR time series was examined, in which the full RR time series includes normal sinus beats as well as any supraventricular and ventricular ectopic beats.

### HRV Analysis: Standard Measures

Standard techniques of HRV analysis are grouped into time and frequency (spectral) domain methods (Heart rate variability 1996). A subset of the former, intended to quantify short-term variability, is based on the difference between consecutive normal-to-normal intervals (ANN, also termed NN increments); the latter on the spectral power of the NN intervals.

The following four traditional time and frequency HRV measures of short-term fluctuations were computed using open-source software (see Goldberger, A. L., Amaral, L. A. N., Glass, L., Hausdorff, J. M., Ivanov, P. C., Mark, R. G., et al. (2000). Physiobank, Physiotoolkit, and PhysioNet: components of a new Research Resource for Complex Physiologic Signals. Circulation 101, e215-e220, herein “Goldberger 2000”) available at the PhysioNet website (www.physionet.org):

Time domain:

(1) pNNx measures: the percentage of ΔNN,>x ms. Here, x=20 and 50 ms was used. See Mietus, J. E., Peng, C.-K., Henry, I., Goldsmith, R. L., and Goldberger, A. L. (2002). The pNNx files: re-examining a widely used heart rate variability measure. Heart 88, 378-380 (herein “Mietus 2002”).

(2) rMSSD (“root mean square of successive differences”): square root of the mean of the squares of ΔNN intervals.

(3) SDSD (“standard deviation of successive differences”): standard deviation of the ΔNN time series.

Frequency domain:

(1) HF (“high frequency”): spectral power of the NN interval time series between 0.15 and 0.4 Hz.

These sets of time and frequency domain measures are widely interpreted to represent cardiac vagal tone modulation (See Heart rate variability 1996, Billman 2011). By comparison, longer time scale fluctuations, are attributable to both sympathetic and parasympathetic influences (See Heart rate variability 1996, Thayer 2010, and Billman, G. E. (2013). The LF/HF ratio does not accurately measure cardiac sympatho-vagal balance. Front Physiol 4, 26 (herein “Billman 2013”) and were, therefore, not considered here.

### HRV Analysis: Nonlinear Dynamical Indices

The following two widely used nonlinear short-term dynamical indices were computed:

(1) Short-term detrended fluctuation analysis (DFA) exponent, α₁. This measure quantifies the correlations properties of a time series. See Peng, C.-K., Havlin, S., Stanley, H. E., and Goldberger, A. L. (1995). Quantification of scaling exponents and crossover phenomena in nonstationary heartbeat time series. Chaos 5, 82-87. (herein “Peng 1995”). The method is based on the assessment of the slope of the linear regression line of the log-log graph of F(n) versus n. The function F(n) is the root-mean-square fluctuation of the integrated and detrended data, computed using windows of length n. For the analysis of heart rate time series, two indices, α₁ and α₂, quantifying short and long-term behavior, respectively, have been proposed. Here, α₁ was focused on, in which α₁ encompasses scales ranging from 4 to 11 beats, inclusively. See Pikkujamsa, S. M., Makikallio, T. H., Sourander, L. B., Raiha, I. J., Puukka, P., Skytta, J., et al. (1999). Cardiac interbeat interval dynamics from childhood to senescence. Circulation 100, 393-399. (herein “Pikkujamsa 1999”). The correlation properties of time series with α≅1.5 are similar to those of Brownian noise. In contrast, time series with α<0.5 are anti-correlated. The former are smoother than the latter.

(2) Sample entropy (SampEn). This measure quantifies the degree of irregularity of a signal. See Richman, J. S. and Moorman, J. R. (2000). Physiological time-series analysis using approximate entropy and sample entropy. Am J Physiol Heart Circ Physiol 278, H2039-H2049. (herein “Richman 2000). A higher SampEn value implies a more irregular, less predictable signal. Sample entropy is the negative of the natural logarithm of the conditional probability that the (m+1)^{th} components of two distinct segments match (∥xᵢ₊ₘ−xⱼ₊ₘ∥<r) within the tolerance r, given that the first m components match within the same tolerance (∥xᵢ₊ₗ−xⱼ₊ₘ∥<r, for 0≤l≤m−1).

### Statistical Analysis

Spearman's rank and Pearson's product-moment correlation coefficients were used to quantify the dependence of: i) the four novel indices of heart rate fragmentation, ii) the traditional measures of short-term HRV, and iii) the two non-linear dynamical indices, short-term DFA exponent α₁ and SampEn, with the participants' age, using the THEW Healthy Subject Database. Statistical significance was set at a p-value<0.05.

Logistic regression analysis methods were used to assess the relationships between presence of CAD and traditional, nonlinear and fragmentation indices in unadjusted models and models adjusted for age and gender. Normalized odds ratios (i.e., the odds ratio for a one standard deviation change in the measure) were reported to facilitate comparisons among various HRV measures.

The area under the receiver operating characteristic (AUC) curve was used to assess the goodness of fit of each model. The likelihood-ratio test was used to compare the goodness of fit of two nested models. All analyses were performed using raw measures except in the case of skewed variables whose logarithmic or quadratic transformation improved the models' goodness of fit. This improvement was only noted in the case of 24-hour and daytime HF, 24-hour and daytime SDSD and nighttime α₁.

### RESULTS

### Changes in Heart Rate Dynamics with the Participants' Age in the Healthy Population

All four fragmentation indices significantly increased with the participants' age, for all tree time periods, using either NN or RR interval time series. Traditional short-term HRV indices significantly decreased with the participants' age, for all three time periods. The fractal α₁ exponent significantly increased with the participants' age during putative sleep time. For the other time periods, linear correlation analysis indicated an inverse relationship. However, in these cases, the Spearman coefficients were not significant. Sample entropy significantly decreased with the participants' age during the putative wake and sleep periods. However, analyses of the 24-hour period did not reveal any significant association between the two variables. The percentage of supraventricular and ventricular premature beats significantly increased with the participants' age (Spearman rₛ=0.27, p=0.004 and rₛ=0.29, p=0.002, respectively).

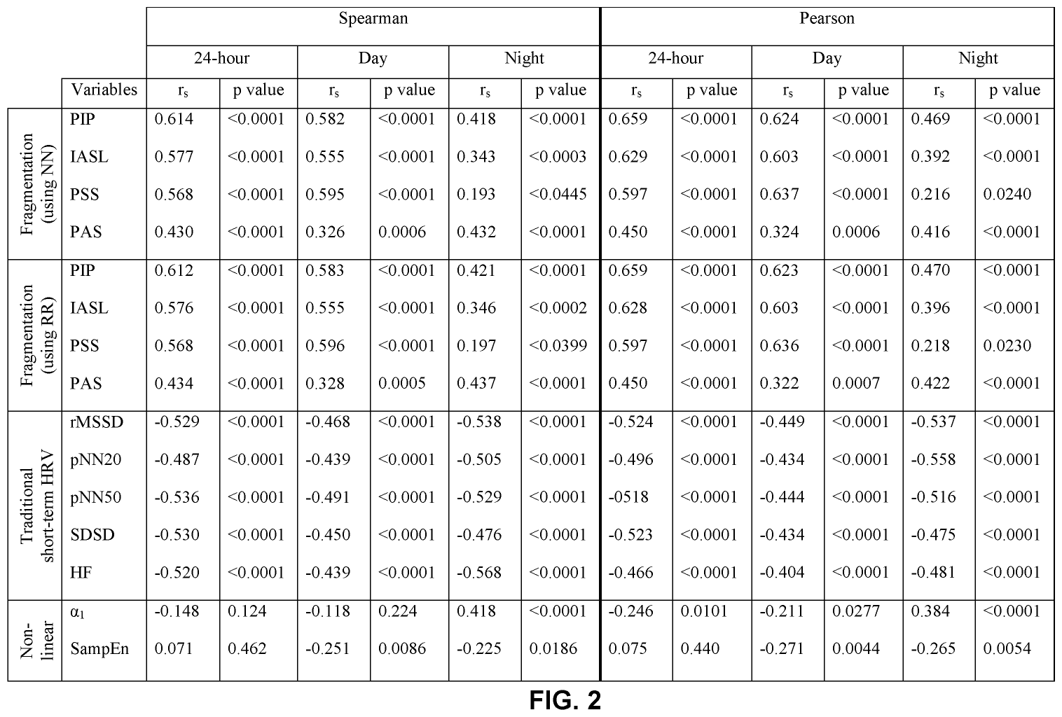

FIG. 2 illustrates a results table of Spearman rank and standardized Pearson product-moment coefficients for the relationships between traditional short-term HRV, nonlinear, and fragmentation indices with cross-sectional age for the group of healthy subjects. The abbreviations used in FIG. 2 are defined by the following: PIP, percentage of inflection points; IALS, inverse of the average length of the acceleration/deceleration segments; PSS, percentage of NN intervals in short segments; PAS, percentage of NN intervals in alternation segments. rMSSD, root mean square of the successive differences; pNN20 and pNN50, percentage of differences between successive NN intervals above 20 ms and 50 ms, respectively; SDSD, standard deviation of successive differences; HF, high frequency spectral power; α₁, detrended fluctuation analysis short-term exponent; SampEn, sample entropy.

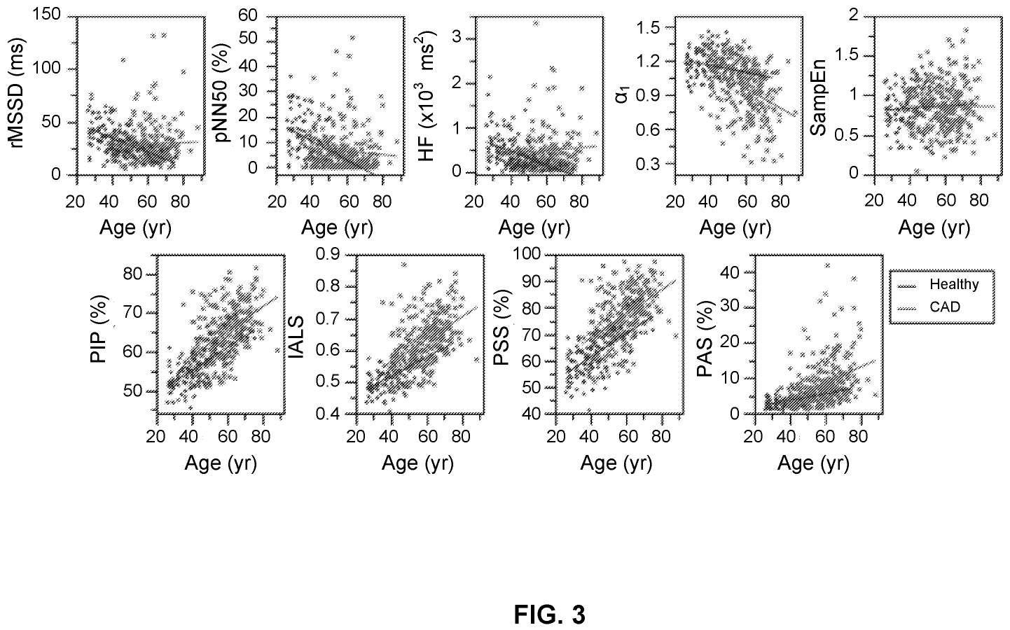

FIG. 3 illustrates scatter plots of the traditional heart rate variability (rMSSD, pNN50 and HF), nonlinear (α₁ and SampEn) and fragmentation (PIP, IALS, PSS and PAS) indices versus the participants' age for the group of healthy subjects and patients with coronary artery disease (CAD), derived from the analysis of the full (˜24-hour) period. The solid lines are the linear regression lines. The abbreviations used in FIG. 3 are defined by the following: rMSSD, root mean square of the successive differences; pNN50, percentage of differences between successive NN intervals above 50 ms; HF, high frequency spectral power; α₁, detrended fluctuation analysis short-term exponent; SampEn, sample entropy; PIP, percentage of inflection points; IALS, inverse of the average length of the acceleration/deceleration segments; PSS, percentage of NN intervals in short segments; PAS, percentage of NN intervals in alternation segments.

### Changes in Heart Rate Dynamics with Coronary Artery Disease

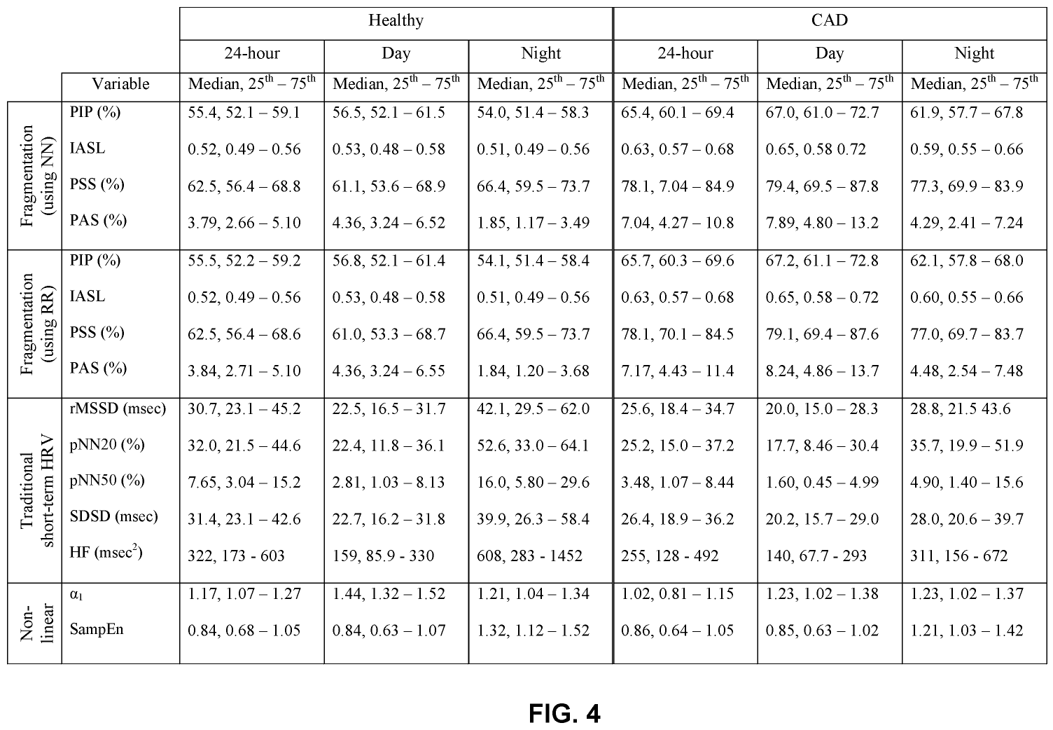

The values (median, 25^{th} and 75^{th} percentiles) of the new fragmentation indices for the groups of healthy subjects and patients with CAD, as well as of the traditional HRV and nonlinear indices, are presented in FIG. 4.

In particular, FIG. 4 illustrates a table of measured values of heart rate variability in healthy subjects and measured values of heart rate variability of subjects with coronary artery disease (CAD), in which the values are reported as median, 25^{th} and 75^{th} percentiles. The abbreviations used in FIG. 4 are defined by the following: PIP, percentage of inflection points; IALS, inverse of the average length of the acceleration/deceleration segments; PSS, percentage of NN intervals in short segments; PAS, percentage of NN intervals in alternation segments. rMSSD, root mean square of the successive differences; pNN20 and pNN50, percentage of differences between successive NN intervals above 20 ms and 50 ms, respectively; SDSD, standard deviation of successive differences; HF, high frequency spectral power; α₁, detrended fluctuation analysis short-term exponent; SampEn, sample entropy

All fragmentation indices significantly (p<0.0001) increased with the participants' age for all time periods in the group of patients with CAD, regardless of using NN or RR time series (FIG. 3). The Pearson correlation coefficients varied between 0.250 and 0.529 for the NN time series and between 0.246 and 0.531 for the RR time series. The correlations for the 24-hour and putative awake periods were relatively stronger than those for the sleep period.

Out of the 15 relationships tested, between each of the five traditional HRV measures and the participants' age, for each of the three time periods, only two were statistically significant. It was found that pNN20 and pNN50 significantly decreased with the participants' age during the putative sleep period. Of the nonlinear indices, only DFA α₁ showed a significant association with participants' age. In this group, α₁ significantly decreased with the participants' age for all time periods.

A one-year increase in age was associated with an increase of 14% in the odds of having CAD (odds ratio=1.14, 95% confidence interval: 1.11-1.17, p<0.0001). The AUC for the model with age as the only covariate was 0.853. Male sex carried a 3.54 fold increase in the odds of CAD (odds ratio=3.54, 95% confidence interval: 2.17-5.78, p<0.0001). The AUC for the null model with age and gender as the sole independent variables was 0.882.

### Unadjusted Analyses

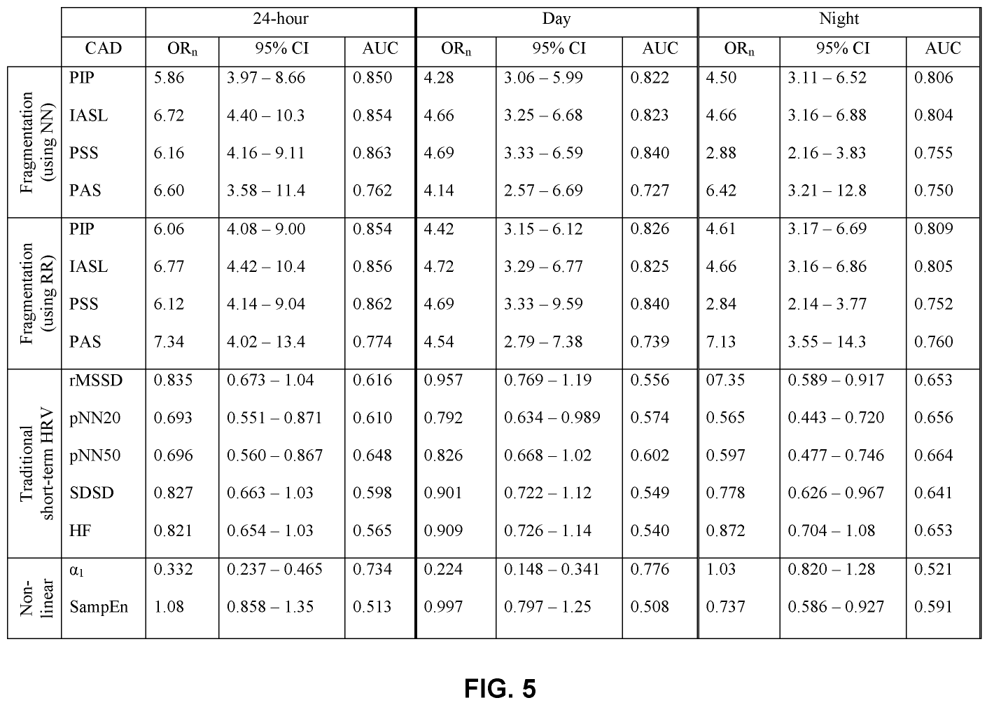

FIG. 5 illustrates a table of values obtained from logistic regression analysis and area under the ROC curve for unadjusted models of CAD, in which the values presented are normalized odds ratio (ORₙ), 95% confidence intervals (95% CI) and area under the receiver operating characteristic curve (AUC). The abbreviations used in FIG. 5 are defined by the following: PIP, percentage of inflection points; IALS, inverse of the average length of the acceleration/deceleration segments; PSS, percentage of NN intervals in short segments; PAS, percentage of NN intervals in alternation segments. rMSSD, root mean square of the successive differences; pNN20 and pNN50, percentage of differences between successive NN intervals above 20 ms and 50 ms, respectively; SDSD, standard deviation of successive differences; HF, high frequency spectral power; α₁, detrended fluctuation analysis short-term exponent; SampEn, sample entropy. The analysis was performed using raw measures except in the case of 24-hour and daytime HF, 24-hour and daytime SDSD and nighttime α₁variables, for which the models with the transformed variables (log in the case of HF and SDSD, and square in the case of α₁) fitted the data better than those with the raw variables.

In the unadjusted analyses (FIG. 5), higher fragmentation indices were significantly associated with presence of CAD, for all time periods, using both NN and RR interval time series. Depending of the specific index and time period considered, a one-standard deviation increase in any of the fragmentation indices was associated with a 2.84 to 7.34 fold increase in the odds of CAD.

In comparison, the traditional short-term time and frequency domain HRV measures were inversely associated with presence of CAD for all time periods. However, only a subset of these measures, rMSSD and SDSD during sleep time, pNN50 during sleep and the 24-hour period and pNN20 for all time periods, were significantly associated with CAD in unadjusted models. Of note, these models consistently performed worse than those with the fragmentation indices. For example, for pNN20, the best performing of the HRV measures, a one-standard deviation increase in the value of this variable was only associated with a 26, 77 and 44% increase in the odds of CAD, for the awake, sleep and 24-hour periods, respectively.

Lower values of α₁, for the awake and 24-hour periods, and of SampEn for the sleep period were also significantly associated with presence of CAD. Overall, α₁ was a stronger correlate of CAD than traditional HRV measures, but not as strong as the fragmentation indices. SampEn, even for the sleep period, was among the weakest correlates of CAD.

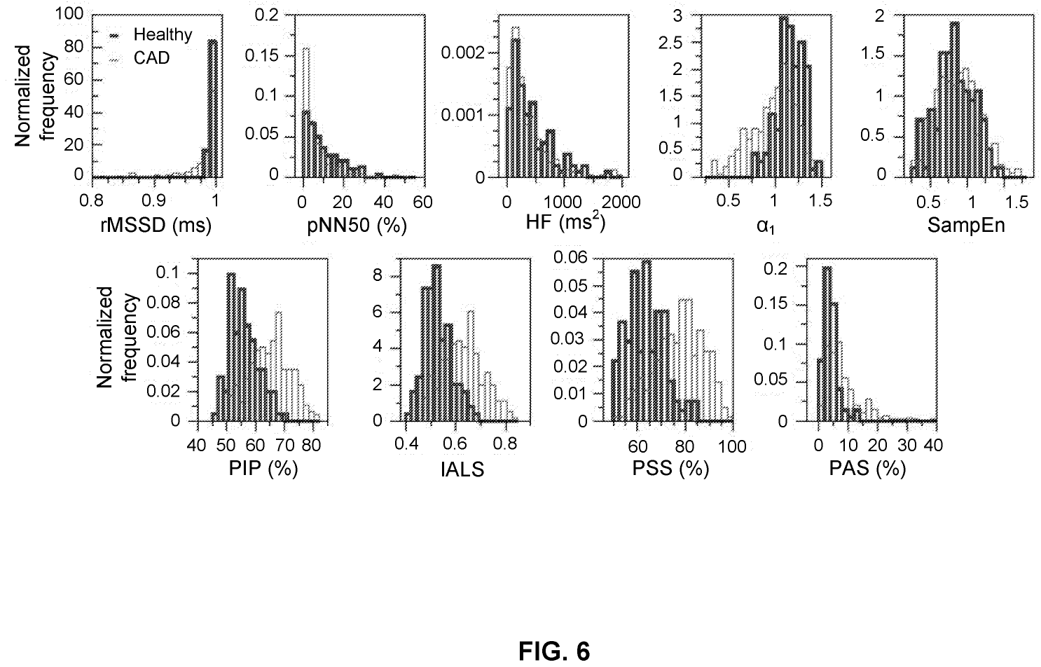

FIG. 6 shows the normalized histograms of the traditional heart rate variability (rMSSD, pNN50 and HF), nonlinear (α₁ and SampEn) and fragmentation (PIP, IALS, PSS and PAS) indices for the groups of healthy subjects and patients with coronary artery disease, for the 24-hour period. The abbreviations used in FIG. 6 are defined by the following: rMSSD, root mean square of the successive differences; pNN50, percentage of differences between successive NN intervals above 50 ms; HF, high frequency spectral power; α₁, detrended fluctuation analysis short-term exponent; SampEn, sample entropy; PIP, percentage of inflection points; IALS, inverse of the average length of the acceleration/deceleration segments; PSS, percentage of NN intervals in short segments; PAS, percentage of NN intervals in alternation segments.

### Adjusted Analyses

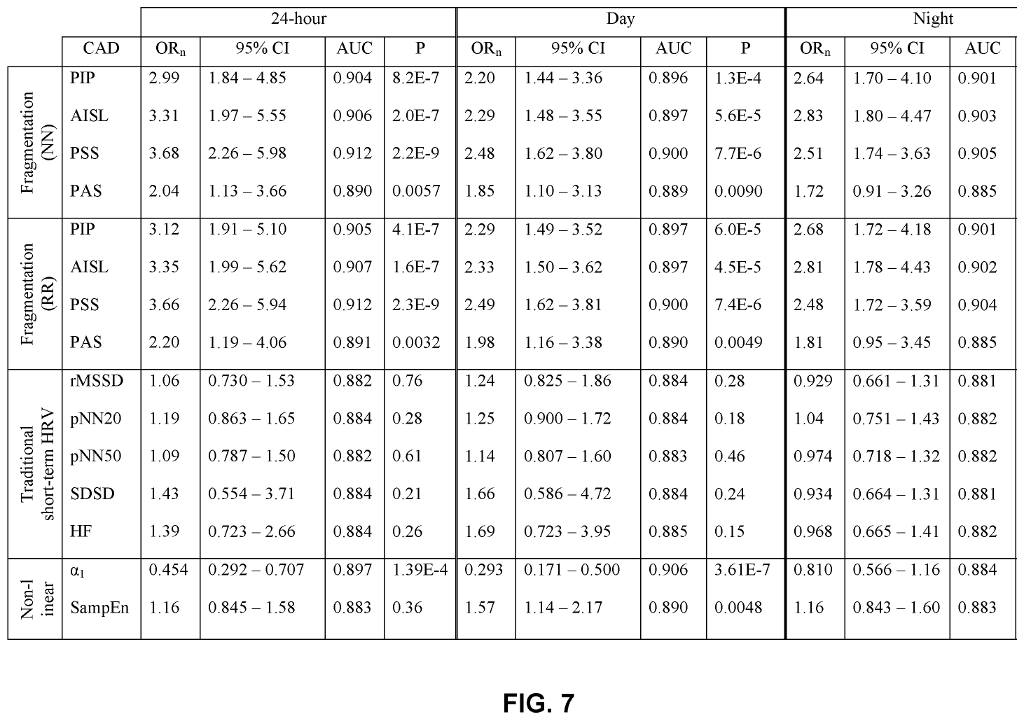

FIG. 7 illustrates a table of values obtained from logistic regression analysis and

AUC for models of CAD adjusted for age and sex, in which the analysis was performed using raw measures. Values presented are the normalized odds ratio (ORₙ) and the 95% confidence intervals (95% CI) for the variables listed in the header column, in models adjusted for age and sex; the area under the receiver operating characteristic curve (AUC) and the p value for the likelihood-ratio test of the null hypothesis that the addition of the HRV measure does not improve the fit of the model with age and sex alone. The abbreviations used in FIG. 7 are defined by the following: PIP, percentage of inflection points; IALS, inverse of the average length of the acceleration/deceleration segments; PSS, percentage of NN intervals in short segments; PAS, percentage of NN intervals in alternation segments. rMSSD, root mean square of the successive differences; pNN20 and pNN50, percentage of differences between successive NN intervals above 20 ms and 50 ms, respectively; SDSD, standard deviation of successive differences; HF, high frequency spectral power; α₁, detrended fluctuation analysis short-term exponent; SampEn, sample entropy.

Fragmentation indices remained positively associated with CAD in models adjusted for age and sex (FIG. 7). Furthermore, the models with any of these indices fitted the data better than the ones with only age and sex, for all time periods, regardless of whether NN or RR time series were used.

Adding any of the fragmentation indices derived from NN interval time series to a model of CAD with the percentages of supraventricular premature beats (% SVPBs) significantly increased its performance (p<0.00001) for all time periods. Specifically, while the AUC for the model with the % SVPBs was 0.754, the AUCs for the models that also included PIP, IALS, PSS or PAS derived from NN intervals time series, were 0.852, 0.856, 0.866 and 0.771, respectively.

None of the associations between traditional time and frequency domain measures and CAD remained significant for any of the time periods when the models were adjusted for age and sex (FIG. 7). For the nonlinear measure α₁, the associations remained significant during the 24-hour and awake periods. For SampEn, the association with CAD during awake was significant, while the relationship during sleep time lost significance. The adjusted models with α₁, for the 24-hour and awake periods, and with SampEn, for the awake period, were superior to the model with only age and sex.

### Relationship between HRV Measures and Mean Heart Rate

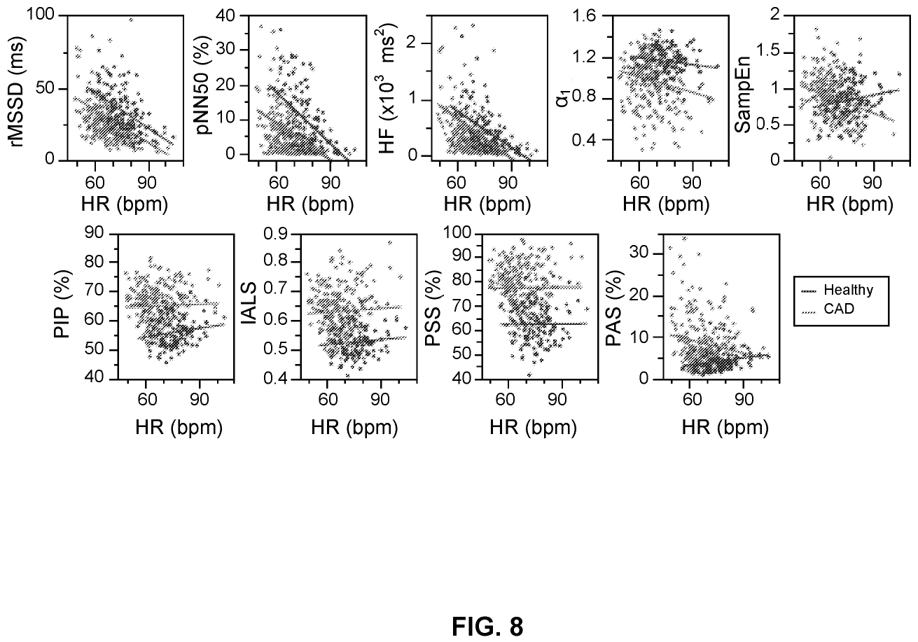

FIG. 8 illustrates scatter plots of the traditional heart rate variability (rMSSD, pNN50 and HF), nonlinear (α₁ and SampEn) and fragmentation (PIP, IALS, PSS and PAS) indices versus mean heart rate (in beats per minute, bpm) for the group of healthy subjects (blue dots) and those with coronary artery disease (CAD, red circles), derived from the analysis of 24-hour NN interval time series. The blue and red lines are the linear regression lines for the healthy and CAD groups, respectively. The abbreviations used in FIG. 8 are defined by the following: rMSSD, root mean square of the successive differences; pNN50, percentage of differences between successive NN intervals above 50 ms; HF, high frequency spectral power; α₁, detrended fluctuation analysis short-term exponent; SampEn, sample entropy; PIP, percentage of inflection points; IALS, inverse of the average length of the acceleration/deceleration segments; PSS, percentage of NN intervals in short segments; PAS, percentage of NN intervals in alternation segments.

In both healthy subjects and those with CAD, the fragmentation indices, PIP, IASL and PSS, were not significantly correlated with mean heart rate (FIG. 8). The same was true for the group of patients with CAD. PAS was weakly correlated with mean heart rate in both healthy subjects and patients with CAD. The correlation was positive, r=0.221 (p=0.021), for the group of healthy subjects and negative, r=−0.146 (p=0.020) in the group of those with CAD.

In contrast, the traditional time and frequency domain measures were strongly correlated with mean heart rate. The standardized Pearson correlation coefficients varied between 0.501 and 0.676 (p<0.0001) in the group of healthy subjects and between 0.111 and 0.574 in the group of patients with CAD (p<0.0001 for all correlations, but the one with SDSD).

SampEn was only weakly correlated with mean heart rate. The correlation was negative in the healthy group, r=−0.185, p=0.054 and positive in the CAD group, r=0.287, p<0.0001. α₁ was positively correlated with mean heart rate in patients with CAD r=0.156, p<0.013. For the healthy subjects, the correlation was not significant.

### DISCUSSION

This study is of potential interest because it presents a new way of assessing short-term HRV under free-running (spontaneous) conditions. The novel methodology and findings are described under the rubric of sinus rate fragmentation (or conversely, smoothness or “fluency”). The conceptual framework is described in the Introduction. The degree of fragmentation of the NN and RR time series, derived from 24-hour Holter monitoring, varied directly as a function of cross-sectional age in a cohort of healthy young to elderly male and female subjects in sinus rhythm. In this group, older age was associated with increased fragmentation. This correlation was noted for the entire 24-hour period, as well as, during putative wake and sleep periods. Furthermore, the fragmentation indices outperformed standard time and frequency domain measures, as well as, two widely used nonlinear measures (DFA α₁ and SampEn), in separating healthy subjects from those with patients with CAD.

The data chosen for analysis in this study was open access Holter data from groups of subjects whose clinical status was well-characterized and presented very sharp population differences: a group of healthy subjects and a group of patients with overt CAD. The healthy subjects were, on average, 20 years younger than the patients with CAD. Therefore, these two group were robustly separated by a model that simply incorporated age and gender (AUC=about 0.88). It was expected that the combination of older age and overt cardiovascular disease in the CAD group to enhance the ability of quantitative methods of HRV to unambiguously discriminate between patients and healthy individuals.

An important link between aging and a variety of overt cardiovascular disease processes is the role of inflammation and fibrosis (See Ghiassian, S. D., Menche, J., Chasman, D. I., Giulianini, F., Wang, R., Ricchiuto, P., et al. (2016). Endophenotype network models: common core of complex diseases. Sci Rep 6, 27414, herein “Ghiassian 2016”), which decrease the amount and/or effectiveness of physiologic vagal tone modulation and promote the breakdown of regulatory networks, such as those controlling heart rate. Therefore, all short-term HRV measures were expected to change in the same directional way with aging and cardiovascular disease, for all time periods. In this regard, and in accord with “canonical” HRV precepts (See Heart rate variability 1996, Billman 2011, Stauss 2003), it was hypothesized that traditional 365 short-term HRV measures would decrease with cross-sectional age in the healthy group and that these measures would be lower for the patients with CAD than healthy subjects. In addition, it was hypothesized that the fragmentation indices would increase with the participants' age in the healthy group and that they would be higher for patients with CAD than healthy subjects.

Strong correlations were found between traditional HRV measures and the participants' age in the healthy group, for all time periods. These findings were in agreement with the generally accepted idea that HRV measures are useful to assess changes in heart rate dynamics with healthy aging (See Pikkujamsa 1999). In contrast, for the group of patients with CAD, traditional HRV indices, with few exceptions, pNN20 and pNN50 during putative sleep, did not significantly change with the participants' age. This counterintuitive finding (e.g., illustrated in FIG. 3) may be due to the confounding effects of heart rate fragmentation, which may increase high-frequency variability not due to physiologic respiratory-vagal modulation. See Domitrovich 2002, Stein 2002, Stein 2005, and Stein 2008.

Furthermore, the ability of traditional short-term HRV measures to separate the healthy and CAD groups was also surprisingly poor. In models adjusted for age and sex, none of the traditional HRV measures significantly discriminated these two groups. Even in unadjusted models, the discriminatory power of conventional HRV measures was not consistent across time periods. In particular, HF power, traditionally interpreted as a measure of vagal tone modulation, did not discriminate the two groups for any of the time periods considered. The results are consistent with previous cautionary reports about the utility of traditional HRV measures to assess vagal tone modulation especially with advanced age or underlying heart disease. See Billman 2013; Burr, R. L. (2007). Interpretation of normalized spectral heart rate variability indices in sleep research: a critical review. Sleep 30, 913-919 (herein “Burr 2007”); Stein 2002; Stein 2005.

The nonlinear indices also did not provide consistent results. For example, α₁ significantly increased with the participants' age during sleep, in both Pearson and Spearman correlation analyses. However, an inverse relationship was found for the awake and 24-hour periods, in Pearson but not in Spearman analyses. In addition, higher α₁ values were significantly associated the presence of CAD during the awake and 24-hour periods, but not during sleep. The degree of randomness of heart rate time series, measured by SampEn, significantly decreased with the participants' age during the awake and sleep periods. Furthermore, while in an unadjusted model, a one standard deviation increase in the degree of randomness of heart rate time series was associated with a 36% decrease in the odds of CAD during sleep time, in a models adjusted for age and sex, a one standard deviation increase in SampEn was associated with a 57% increase in the odds of CAD during awake period. For the other time periods, the associations were not significant.

The inconsistencies of the two nonlinear methods, α₁ and SampEn, are not entirely unexpected. For example, the fluctuation function, F(n), in DFA analysis of 24-hour NN interval time series usually presents a crossover separating “short-term” from “longer-term” behavior. However, the scale at which that crossover occurs may vary substantially from subject to subject. In addition, the degree of linearity of F(n) also tends to vary from subject to subject. SampEn, on the other hand, can be affected by nonstationarities that are common in real world data. In addition, fragmented time series can be highly predictable (leading to low SampEn), as in the case of those with a high density of alternation, or highly irregular, as in the case of “erratic” sinus rhythm (leading to increased SampEn).

The limitations of traditional and newer HRV methods, exemplified by the results reported above and those described by other investigators, help motivate the on-going searches for alternative approaches. See Billman 2013, Burr 2007, Stein 2002, and Stein 2005. The introduction of the concept of fragmentation of heart rate dynamics, accompanied by a set of metrics for its quantification, may be part of this exploration.

Speculatively, possible mechanisms of the observed fragmentation include the breakdown of one or more components of the regulatory network controlling heart rate dynamics. An obvious first question would be whether the higher fragmentation values in CAD versus the healthy group could simply be due to supra-ventricular premature beats (SVPBs) mislabeled as normal sinus beats. While the THEW website describes that three lead Holter monitor recordings were first processed using an automated beat annotation program and then subjected to visual review and adjudication, the possibility that some of the beats labeled as N are actually subtle SVPBs, and not sinus beats, cannot be absolutely excluded. To address this possible confounder, one would assume that the recordings with the highest likelihood of containing hidden SVPBs would be those with the highest percentage of labeled SVPBs. Therefore, to assess whether the results were likely a consequence of mislabeled SVPBs, the performance of a model of CAD with the % SVPBs, as the sole independent variable was compared to that of a model with the % SVPBs and each fragmentation index. It was found that all fragmentation indices added significant information to the model with % SVPBs. This finding supports the contention that fragmentation is not a surrogate measure of “hidden” supraventricular ectopy.

A second question would be whether abnormalities in breathing dynamics could be responsible for the fragmentation of heart rate dynamics through respiratory-cardiac coupling. Without a direct measure of respiration, the possibility that inter-breath interval time series were themselves fragmented might not be excluded. However, such a mechanism is unlikely since beat-to-beat changes in the sign of heart rate acceleration are above the frequency response of the vagal-sinus modulatory system. In fact, the coupling between the sinus node and the vagus tends to drop-off at high respiratory rates. See Angelone 1964 and Hirsch 1981. Furthermore, the most erratic variants of sinus rhythm are seen in the populations of older healthy subjects and those with organic heart disease (see Stein 2005 and Stein 2008) groups with the most impaired vagal modulatory capacity and, therefore, those least likely to show very high frequency coupling between autonomic and electrophysiologic components.

The specific electrophysiologic bases for fragmentation of heart rate dynamics remain to be determined. More than one mechanism may be contributory. For example, alternans phenotypes could be due to sinus node exit block or to very subtle atrial bigeminy with SVPBs originating near or even within the sino-atrial (SA) node (see Geiger 1945). Another mechanism that could account for alternation would be modulated sinus node parasystole (see Jalife, J. (2013). Modulated parasystole: still relevant after all these years. Heart Rhythm 10, 1441-1443, herein “Jalife 2013”), an arrhythmia in which two pacemaker sites in the SA area show bidirectional coupling and appear to “compete” for control of the heartbeat. Under certain parameter regimes, such coupling has been shown, both experimentally and theoretically, to induce alternans (bigeminal) type patterns (Hirsch 1981). The underlying electrophysiologic mechanisms to account for fragmentation may also involve perturbations of internal pacemaker “clocks” in the SA node. See Lakatta, E. G., Maltsev, V. A., and Vinogradova, T. M. (2010). A coupled SYSTEM of intracellular Ca2+ clocks and surface membrane voltage clocks controls the timekeeping mechanism of the hearts pacemaker. Circ Res 106, 659-673 (herein “Lakatta 2010”). From a pathophysiologic viewpoint, mechanisms related to altered conduction and/or abnormal automaticity all reflect in stabilities in the parasympathetic-SA node-atrial network. Given this substrate of instability, highly fragmented (whether erratic or periodic) types of NN patterns may represent pre- or even pro-arrhythmic markers. The findings that fragmentation is increased in the elderly and in those with established CAD support this contention. Fragmentation would be of high interest if it were a forerunner of arrhythmias such as atrial fibrillation or other tachyarrhythmias in which the control network becomes so unstable that sinus node function is overridden by ectopic stimuli. Whether fragmentation is an independent risk marker of cardiovascular mortality related to heart failure also remains to be determined, as does any relationship to classical sick sinus syndrome.

More generally, the findings here support a modification in the standard classification of sinus rhythm into “phasic” and “non-phasic” variants. See Faulkner, J. M. (1930). The significance of sinus arrhythmia in old people. Am J Med Sci 180, 42-46 (herein “Faulkner 1930”); Fisch, C. and Knoebel, S. (2000). Electrocardiography of clinical arrhythmias (Wiley-Blackwell) (herein “Fisch 2000”); Hirsch 1981. The first category refers to the oscillations in heart rate that are coherent with respiration and are most marked in younger individuals at rest, during deep sleep or with meditation (classic RSA). Non-phasic sinus arrhythmia, a term that has largely disappeared from the clinical lexicon, has been used to refer to a variety of sinus variants without this strict periodicity, including erratic sinus rhythm, and usually connotes abnormal sinus function (Stein 2005). However, non-phasic types of sinus arrhythmia may also occur as physiologic variants, e.g., during exercise and recovery. An alternative schema would be classify sinus rhythm into phasic and non-phasic types, and then sub-divide non-phasic into either physiologic due to short term trends but without tight respiratory coupling and non-physiologic, i.e., fragmented categories. However, fragmentation analysis per se may not separate phasic and non-phasic variants into two discrete bins. Rather, it quantifies, in a continuous way, the degree to which fragmentation is present.

Heart rate fragmentation may account for some of the abnormal patterns in Poincaré maps previously reported. See Brouwer, J., van Veldhuisen, D. J., Man in 't Veld, A. J., Haaksma, J., Dijk, W. A., Visser, K. R., et al. (1996). Prognostic value of heart rate variability during long-term follow-up in patients with mild to moderate heart failure. The Dutch Ibopamine Multicenter Trial Study Group. J Am Coll Cardiol 28, 1183-1189 (herein “Brouwer 1996); Domitrovich 2002; Gladuli, A., Moise, N. S., Hemsley, S. A., and Otani, N. F. (2011). Poincare plots and tachograms reveal beat patterning in sick sinus syndrome with supraventricular tachycardia and varying AV nodal block. J Vet Cardiol 13, 63-70 (herein “Gladuli 2011”); Huikuri, H. V., Seppanen, T., Koistinen, M. J., Airaksinen, J., Ikaheimo, M. J., Castellanos, A., et al. (1996). Abnormalities in beat-to-beat dynamics of heart rate before the spontaneous onset of life threatening ventricular tachyarrhythmias in patients with prior myocardial infarction. Circulation 93, 1836-1844 (herein “Huikuri 1996”); Stein 2005; Stein 2008; Woo, M. A., Stevenson, W. G., Moser, D. K., Trelease, R. B., and Harper, R. M. (1992). Patterns of beat-to-beat heart rate variability in advanced heart failure. Am. Heart J. 123, 704-710 (herein “Woo 1992”). Such maps contain important information about the temporal structure of a time series. However, they are difficult to quantify. Commonly employed metrics such as SD1, SD2 and SD1/SD2, only measure linear properties of the data that are also captured by time domain HRV measures such as rMSSD and SDSD. See Brennan, M., Palaniswami, M., and Kamen, P. (2001). Do existing measures of Poincare plot geometry reflect nonlinear features of heart rate variability? IEEE Trans Biomed Eng 48, 1342-1347 (herein “Brennan 2001”). If heart rate fragmentation is found to be one of the mechanisms underlying such abnormal patterns, the metrics introduced here may help identify, in a fully automated way, the time series associated with certain types of anomalous Poincare plots.

Additional fragmentation-related indices may also prove useful. Examples include the densities of: (i) “hard edges,” defined as inflection points for which ΔNNᵢ×ΔNNᵢ₊₁≤0; (ii) “soft edges,” defined as ΔNNᵢ×ΔNNᵢ₊₁=0, where ΔNNᵢ≠ΔNNᵢ₊₁; (iii) “short segments,” defined as acceleration/deceleration segments encapsulated between “hard edges” or “soft edges;” and (iv) segments for which heart rate does not change.

Finally, the fragmentation indices have a number of attractive features. First, the fragmentation indices are independent of the mean heart rate (e.g., illustrated in FIG. 8). The only exception is PAS, which is not a general fragmentation index, but quantifies a particular type of fragmentation (pattern of the type “ABAB”, where “A” and “B” represent increments of opposite sign). In contrast, traditional short-term time and frequency domain measures showed highly significant negative associations with mean heart rate, both in the group of healthy subjects and of those with CAD. These results are in line with those reported in other studies. See Monfredi, O., Lyashkov, A. E., Johnsen, A. B., Inada, S., Schneider, H., Wang, R., et al. (2014). Biophysical characterization of the underappreciated and important relationship between heart rate variability and heart rate. Hypertension 64, 1334-1343 (herein “Monfredi 2014”); Sacha, J. (2014). Interaction between heart rate and heart rate variability. Ann Noninvasive Electrocardiol 19, 207-216 (herein “Sacha 2014”). Second, by construction, the fragmentation indices (including PAS) are also independent of the amplitude of the time series. This feature is due to the fact that accelerations/decelerations were defined as increments/decrements in heart rate of any magnitude. Thus, two time series with fluctuation patterns that only differ in amplitude (e.g., time series, uᵢ and vᵢ for which uᵢ/vᵢ=c, where c is a constant) will have exactly the same degree of fragmentation.

Future studies may be needed to explore whether the use of a threshold>0 in the definition of accelerations and decelerations further increases the discriminatory power of these measures in HRV analyses. Third, the fragmentation indices are among the measures least affected by nonstationarities. The reason is that the operation of calculating the increment time series, used to detect the inflection points (i.e., changes in heart rate acceleration sign), detrends the data. Fourth, the fragmentation indices can be computed using NN or RR interval time series. Indeed the use of the latter did not impair the discriminatory power of the fragmentation indices for the populations studied here. This finding, if validated, may facilitate fully automated implementations of the method. Future studies will also help determine the effect of data length on the confidence intervals of the fragmentation indices.

### EXAMPLE 1—HEART RATE FRAGMENTATION: A SYMBOLIC DYNAMICAL APPROACH

Analysis of fluctuations in cardiac interbeat intervals, under the rubric of heart rate variability (HRV), continues to generate much interest as a uniquely accessible window into the complex network of regulatory mechanisms controlling the sino-atrial (SA) node (see Heart rate variability 1996; Billman 2013). Particular emphasis has been placed on the analysis of short-term fluctuations, i.e., oscillatory patterns with cycle lengths ranging from approximately four to eight consecutive beats. Such fluctuations, termed respiratory sinus arrhythmia, are primarily ascribable to the coupling between heart rate and breathing, mediated by the parasympathetic (vagal) nervous system (see Angelone 1964; Hirsch 1981).

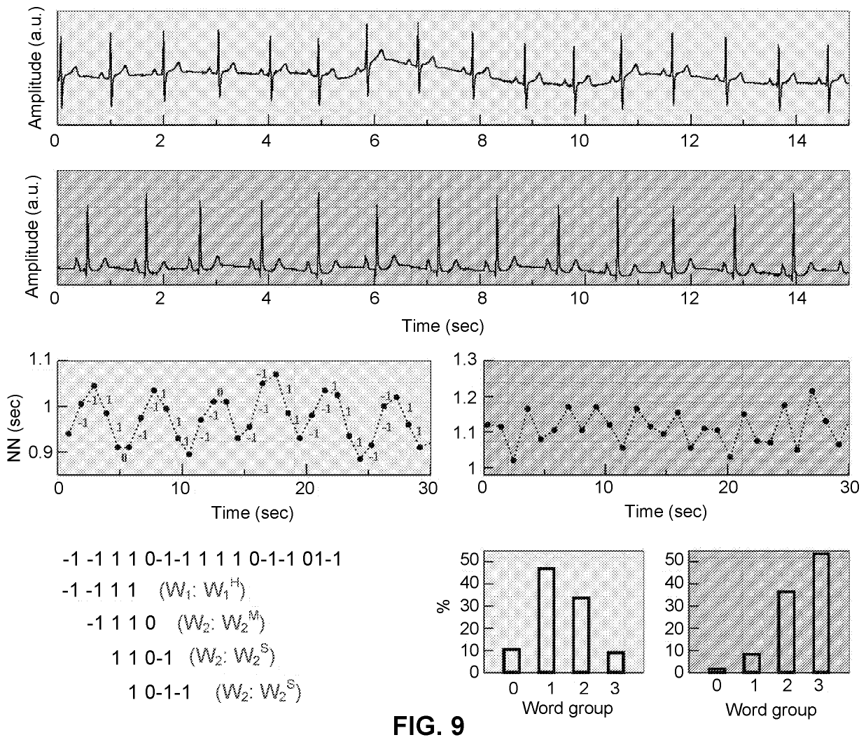

However, short-term fluctuations in heart rate are not always a marker of healthy cardiopulmonary interactions (FIG. 9). See Makika

Ilio, T. H., Koistinen, J., Jordaens, L., Tulppo, M. P., Wood, N., Golosarsky, B., et al. (1999). Heart rate dynamics before spontaneous onset of ventricular fibrillation in patients with healed myocardial infarcts. Am. J. Cardiol. 83, 880-884 (herein “Makikallio 1999”); Domitrovich 2002; Stein 2002; Wiklund, U., Hornsten, R., Karlsson, M., Suhr, O. B., and Jensen, S. M. (2008). Abnormal heart rate variability and subtle atrial arrhythmia in patients with familial amyloidotic polyneuropathy. Ann. Noninvasive Electrocardiol. 13, 249-256. (herein “Wiklund 2008”); Costa I 2017. They may also be associated with abnormalities in the function of the neuroautonomic system, the SA node and other electrophysiologic components (Geiger 1945; Binkley 1995; Jalife2013). Anomalous short-term variability is important for two major reasons: (1) it may confound the assessment of vagal tone modulation using conventional time and frequency domain HRV measures, leading to inflated estimates of “healthy” autonomic function in the elderly and especially in those with clinical or pre-clinical organic heart disease; and (2) its presence, itself, may be a novel dynamical biomarker of pathology and increased risk of adverse cardiovascular outcomes.

To help further address these issues, Costa I 2017 recently introduced the concept of heart rate fragmentation, along with a set of metrics to quantify this property. The underlying framework is based on the observation that sustained physiologic changes in heart rate cannot persist at frequencies higher than those at which the intact parasympathetic nervous system operates. Although the maximal physiologic response frequency is difficult to pinpoint, anticorrelated, beat-to-beat changes in heart rate, characterized by frequent changes from acceleration to deceleration and vice versa, are clearly atypical or frankly abnormal. The fragmentation indices that were introduced (Costa I 2017) quantify the density of this type of pattern. The assumption was that the systems manifesting the highest degree of fragmentation (loss of “fluency”) were the most pathologic ones.

Costa I 2017 showed that: (i) the degree of fragmentation of the NN and RR time series, derived from 24-h Holter monitoring, varied directly as a function of cross-sectional age in cohorts of healthy young to elderly male and female subjects in sinus rhythm and of those with coronary artery disease (CAD); (ii) the degree of fragmentation was significantly higher in patients with CAD than in healthy subjects, both in unadjusted models and in those adjusted for age and sex; and (iii) fragmentation indices outperformed standard time and frequency domain measures, as well as, widely used non-linear measures, in separating healthy subjects from patients with CAD.

To gain additional insight into the temporal structure of heart rate fragmentation, a symbolic dynamical approach to the quantification of this property is now introduced. In general, symbolic mapping deliberately reduces the overall information content of a signal. At the same time, it provides a useful way of highlighting certain features deemed of interest, while deemphasizing others. In heart rate fragmentation studies, examples of features of interest are the changes in heart rate acceleration sign, whereas “details” that one may choose to ignore are the magnitudes of those changes. In this study, the general hypotheses were that the degree of fragmentation, quantified by a set of variables derived from the symbolic dynamical analysis described below, would be: (1) higher in older subjects than in their younger counterparts, and (2) higher in patients with CAD than in healthy subjects. In addition, the present disclosure seeks to explore whether different symbolic “phenotypes” could help in distinguishing physiologic aging from aging in the context of overt organic heart disease.

### Methods

### Databases

Two long-term (-24-h) ECG ambulatory databases from the Intercity Digital Electrocardiogram Alliance (IDEAL) study were employed (Costa I 2017). The de-identified recordings are made available via the University of Rochester Telemetric and Holter ECG Warehouse (THEW) archives (http://thew-project.org/databases.htm).

1. Healthy Subjects Database (THEW identification: E-HOL-03¬0202-003). The database comprises 24-h Holter recordings from 202 ostensibly healthy subjects (102 males). The ECG signals were recorded at a sampling frequency of 200 Hz. Automated beat annotations were manually reviewed and adjudicated. The following subjects were excluded: 45 subjects with more than 2% non-sinus beats, 37 younger than 25 years old, 10 with BMI>30 Kg/m2 and one with <12 h of data. Overall, the analyses included 30 Kg/m2 and one with <12 h of data. Overall, the analyses included 109 healthy adult subjects (60 male), age (median, 25th-75th) 40, 33-49 years.

2. Coronary Artery Disease Subjects Database (THEW identification E-HOL-03-0271-002). This database comprises 24-h Holter recordings from 271 patients (223 males). Subjects had an abnormal coronary angiogram (at least one vessel with luminal narrowing>75%) and either exercise-induced ischemia or a documented previous myocardial infarction. For the analysis, 11 subjects whose Holter recordings contained≥20% non-sinus beats and four subjects with less than 12 h of data were also excluded. Overall, analyzed 256 subjects were analyzed: (208 male), age (median, 25th-75th): 60; 51-67 years; left ventricular ejection fraction 56.5%, 50-66.

As previously described in Costa I 2017, presumed waking and sleeping periods were estimated as the six consecutive hours of highest and lowest heart rates, respectively. These periods were calculated from the NN interval time series using a 6 h moving average window shifted 15 min at a time. From the continuous ECG of each subject, the time series of the RR and NN intervals were derived. The former is the sequence of intervals between consecutive QRS complexes. The latter, is the subset of intervals between consecutive normal sinus to normal sinus QRS complexes.

### Symbolic Mapping and Dynamical Analysis

The original interbeat interval time series, {sᵢ}, 1≤i≤N (N, time series length) was mapped to a ternary symbolic sequence as follows: “−1” if ΔNNᵢ<0, “0” if ΔNNᵢ=0, and “1” if ΔNNᵢ>0. Of note, since the ECG signals were sampled at 200 Hz, the resolution (t) of both the NN interval and the increment time series was 5 ms ( 1/200 s). Taking the sampling frequency into consideration, the symbolic mapping rules can be alternatively written as: “1” if ΔNNᵢ<−5 ms, “0” if −5<ΔNNᵢ<5 ms, and “−1” if ΔNNᵢ>5 ms. Next, the percentages of different segments of 1 consecutive symbols, wᵢ=(sᵢ, sᵢ₊₁, . . . , sᵢ₊₁₋₁), 1<i<N−1+1, termed “words,” were calculated. (With an alphabet of n symbols, the number of different words of length l is n¹.) Words derived from the NN interval time series were termed NN words. Words derived from the RR interval time series were termed RR words.

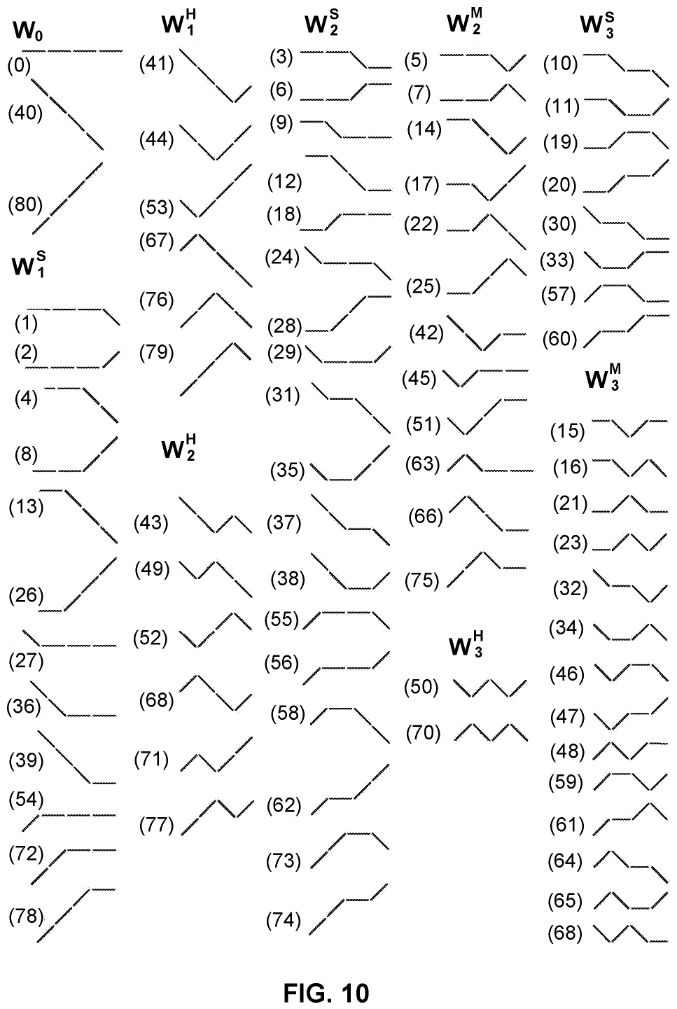

In order to focus on the analysis of short-term dynamical patterns occurring at the respiratory frequency, a word length of four was chosen, which corresponds to time scales of approximately 3-5 s, depending on the heart rate. Subsequently, the words were grouped according to the number and type of transition between consecutive symbols (FIG. 9). Reversals in heart rate acceleration (ΔNNᵢ×ΔNNᵢ₊₁<0), i.e., transitions from symbol “1” to “−1” or vice versa, were termed hard (H) inflection points. Transitions to or from zero acceleration (ΔNNᵢ×ΔNNᵢ₊₁=0, ΔNNᵢ≠ΔNNᵢ₊₁), i.e., transitions from symbols “1” or “−1” to “0”, or vice versa, were termed soft (S) inflection points. The higher the number of inflection points in a word the more fragmented it was. Words of length four can contain no more than three inflection points. Word groups with only hard, only soft and a combination of hard and soft inflection points were, respectively, labeled Wⱼ^{H}, Wⱼ^{S}, and Wⱼ^{M} (where “M” stands for “mixed” and j indicates the number of inflection points). The word groups with more than one inflection point, Wⱼ (2<j<3), for which the type of inflection point was not specified, included the words from subgroups Wⱼ^{H}, Wⱼ^{S}, and Wⱼ^{M}. The word group W₁ included the words from subgroups W₁^{H}, W₁^{S}. FIG. 10 shows a schematic representation of all the different words (n=81).

Of note, to calculate the percentage of each NN word, two different denominators can be used: the total number of NN words and the total number of RR words. The former is not affected by the presence of ectopic beats, while the latter takes them into consideration. Here, the percentages of W₀, Wⱼ, Wⱼ^{H}, Wⱼ^{S}, and Wⱼ^{M}, 1<j<3 were computed using the total number of NN words. In addition, the percentages of hard (soft) NN words that contained one, two and three hard (soft) inflection points were calculated. In these cases, the denominators were the total number of hard (soft) NN words with at least one inflection point. These word subgroups were labeled Wⱼ^{H}* (Wⱼ^{S}*). Thus, while Wⱼ^{H}, (Wⱼ^{S}) represents the overall percentage of words with j hard (soft) inflection points, Wⱼ^{H}* (Wⱼ^{S}*) represents the percentage of hard (soft) words with j inflection points. They were calculated as follows: Wⱼ^{H}*=Wⱼ^{H}/Σᵢ₌₁³Wᵢ^{H}(Wⱼ^{S}*=Wⱼ^{S}*/Wⱼ^{S}/Σᵢ₌₁³Wᵢ^{S}).

The percentages of hard (PIP^{H}) and soft (PIP^{S}) inflection points were also computed. PIP^{H} and PIP^{S} are subcategories of the fragmentation index, PIP, previously introduced (Costa I 2017).

The analysis also included determining how PIP^{H}, PIP^{S} and the different group of words changed with the participants' age and with disease in unadjusted and adjusted [for age and sex, and age, sex and average NN interval (AVNN)] logistic models. Taking into consideration that heart rate fragmentation has been shown (Costa I 2017) to increase with cross-sectional age and with CAD in these databases, it was hypothesized that the percentages of words in groups W₀ and W₁ (least fragmented), would decrease with the participants' age and with disease, while the percentages of words in groups W₂ and W₃ (most fragmented), would increase, regardless of the type of inflection points.

### Statistical Analysis

Outcome variables were summarized by their median, 25th and 75th percentile values.

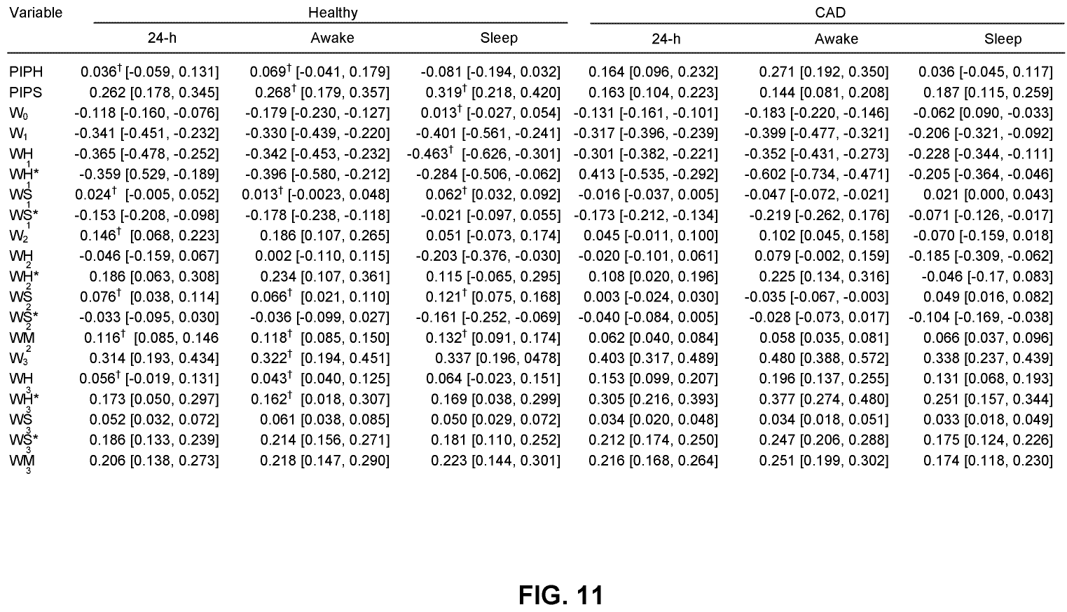

Linear regression models were used to quantify the dependence of each of the outcome variables (y: Wⱼ, Wⱼ^{H}, Wⱼ^{H}*, Wⱼ^{S}, Wⱼ^{S}*, Wⱼ^{M}, PIP^{H} and PIP^{S}) with the participants' age. These models included the interaction term between age and sample population to assess whether the regression slopes were the same in the two populations. In addition, these models included AVNN to control for the effects of this variable on each of the outcome variables (y=c+β₁×age+β₂×population+β₃×age×population+β₄×AVNN, where c is a constant). (The values of β₁ and of β₁+β₃ along with their confidence intervals are provided in FIG. 11 for the groups of healthy subjects and those with CAD, respectively.) Statistical significance was set at a p<0.05.

Logistic regression analysis was used to assess the relationships between presence of CAD and each of the outcome measures in unadjusted models, and models adjusted for age and sex, and age, sex, and AVNN. To facilitate comparisons among various outcome variables, normalized odds ratios (i.e., the odds ratio for a one standard deviation change in a given measure) were reported.

The area under the receiver operating characteristic (AUC) curve was used to assess the discrimination of each model. The likelihood ratio test was used to compare the fit of two nested models. All analyses were performed using raw measures.

### Results

### Relationship between Heart Rate Fragmentation Indices and Participants' Age

As previously reported in Costa I 2017, the overall percentage of inflection points (soft and hard combined) significantly increased with the participants' age in both healthy subjects and those with CAD. However, the percentages of only soft and only hard inflection points changed with the participants' age in different ways for each of the groups (FIG. 11). In healthy subjects, the percentage of soft inflection points significantly increased with the participants' age (slope of the regression line, [95% CI]: 0.26 [0.18, 0.35] %/yr), while the percentage of hard inflection points did not (0.04 [−0.06,0.13] %/yr). In patients with CAD, both, the percentages of soft and hard inflection points increased with the participants' age at identical rates, 0.16 [0.10, 0.22] %/yr and 0.16 [0.10, 0.23] %/yr, respectively. These findings were consistent across all time periods with only one exception. During the putative sleep period, the increase in the percentage of hard inflection points did not reach statistical significance for those with CAD.

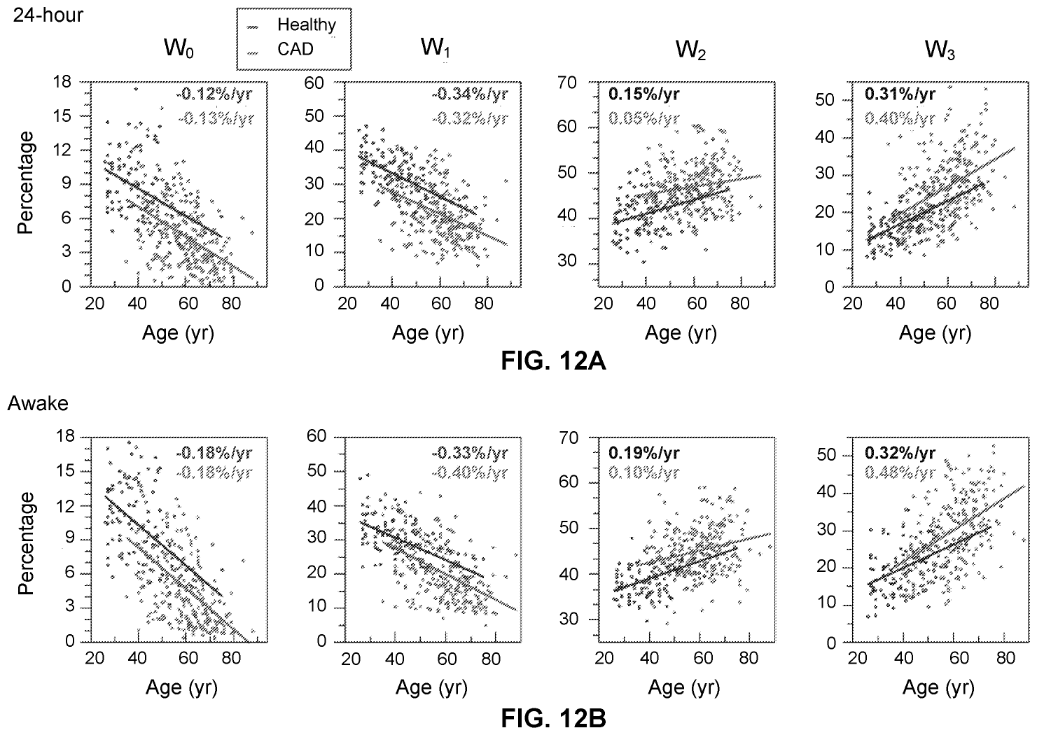

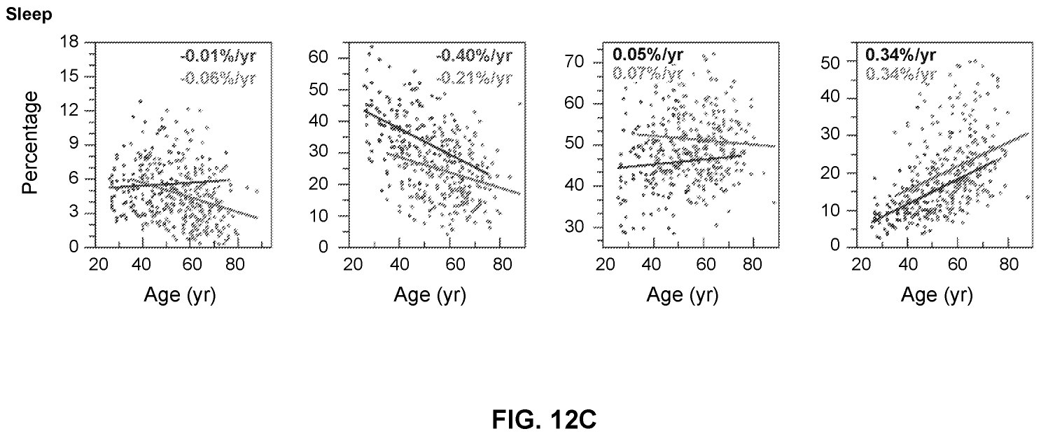

Overall, the percentage of words W₀ and W₁, i.e., the least fragmented (most “fluent”), decreased with the participants' age both in the groups of healthy subjects and those with CAD. All relationships were significant with the exception of the one with W₀ during the putative sleep period for the healthy subjects. Complementarily, the percentage of words W₃, the most fragmented (least “fluent”) significantly increased with the participants' age in both sample populations for all time periods (FIG. 11 and FIGS. 12A-12C). The percentage of words W₂ that capture patterns of transitions between fluent and fragmented dynamics also tended to increase with the participants' age. However, only some of these relationships reached significance.

In both healthy subjects and patients with CAD, the word groups with the strongest association with the participants' age were W₁, among the most fluent, and W₃, among the most fragmented. In both cases, the magnitude of the rate of change was above 0.2%/yr (negative rate for W₁ and positive rate for W₃).

In general, the number of inflection points in a given word subgroup, not the type of inflection points (H, S or M) determined the directionality of the changes in its density with the participants' age. Among a total of 84 relationships (14 word subgroups, three time periods and two sample populations) only 7 did not change with the participants' age in the expected direction (FIG. 11); all except one of these relationships (W₂^{S} for the group of patients with CAD) were for the putative sleep period.

The most notable difference between healthy subjects and patients with CAD in the symbolic analysis related to the words with three hard inflection points (W₃^{H}). While the percentage of these words did not significantly change with cross-sectional age in the group of healthy subjects for any time periods, it significantly increased in the group of patients with CAD, for all time periods, at rates varying between 0.13 and 0.20%/year.

### Changes in Heart Rate Fragmentation Indices with Coronary Artery Disease

A 1-year increase in age was associated with an increase of 14% in the odds of having CAD (odds ratio [95% CI]: 1.14 [1.11, 1.17]<0.0001). The AUC for the model with age as the only covariate was 0.853. Male sex carried a 3.54-fold increase in the odds of CAD (3.54 [2.17, 5.78], p<0.0001). The AUC for the null model with age and sex as the sole independent variables was 0.882. The AUC for the null model with age, sex and AVNN was 0.910.

### 1. Unadjusted Analyses

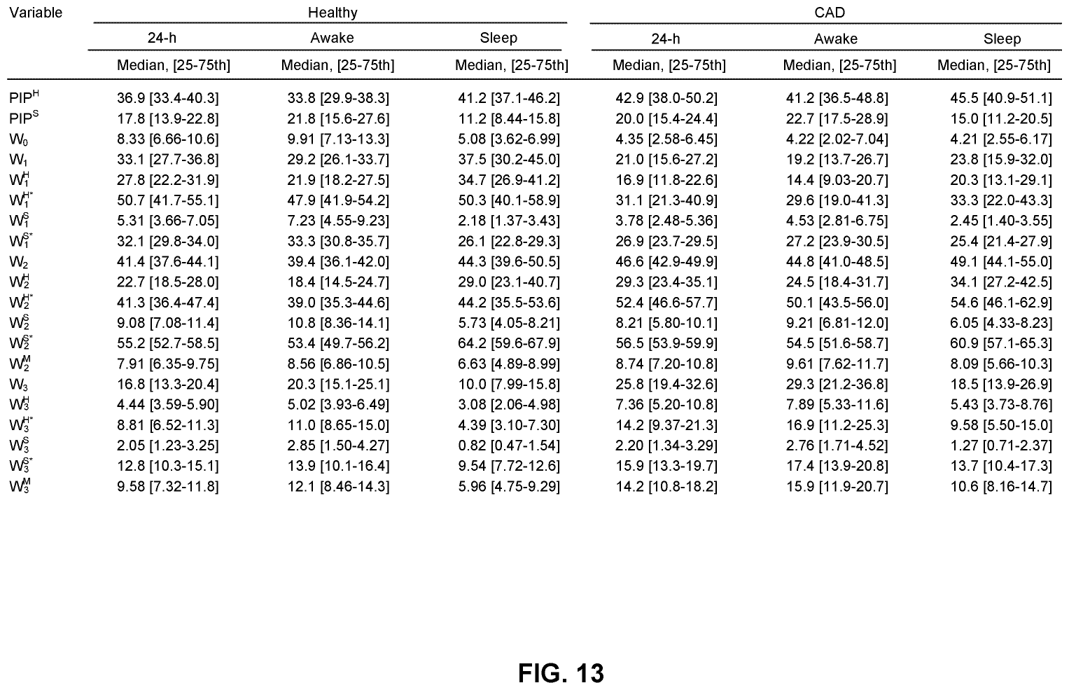

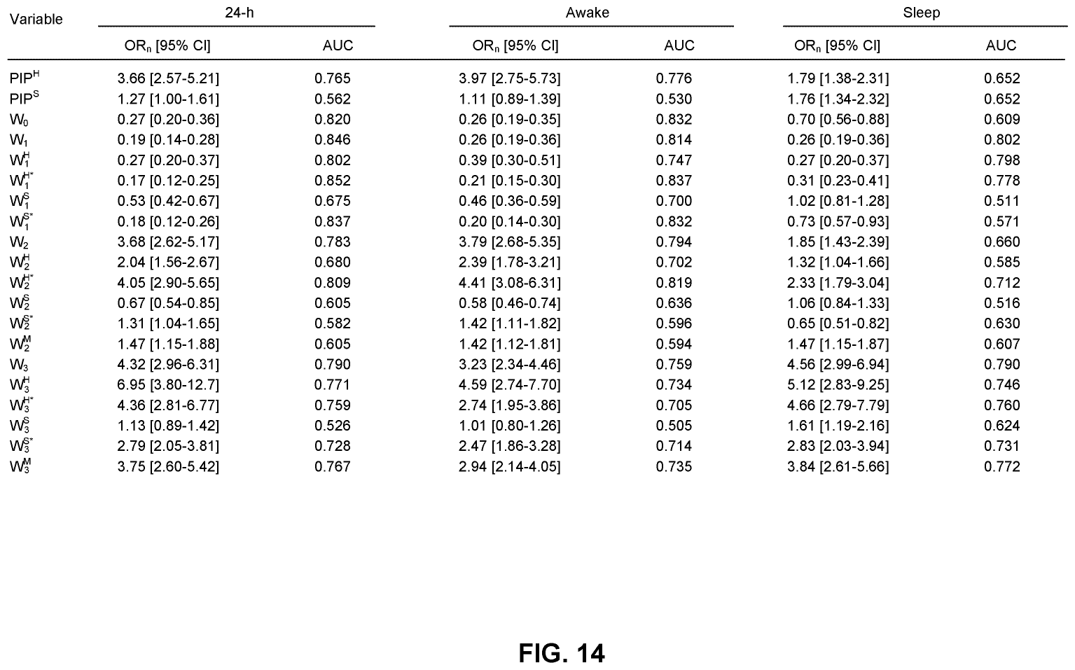

Summary statistics of the calculated indices for the groups of healthy subjects and patients with CAD are presented in FIG. 13. In unadjusted analyses (FIG. 14), the percentage of hard inflection points, PIP^{H}, was significantly higher in those with CAD than in healthy subjects for the three time periods.

The percentage of soft inflection points, PIP^{s}, showed a similar behavior. However, statistical significance was only reached for the putative sleep time.

In these models, lower percentages of words without (W₀) and with only one (W₁) inflection point, and higher percentages of words with two (W₂) and three (W₃) inflection points were significantly associated with the presence of CAD, for all time periods. Similar results were obtained for the subgroups of hard (Wⱼ^{H} and Wⱼ^{H}*) and mixed (Wⱼ^{M},) words with any number of inflection points, for all time periods.

The percentages of words with one and two soft inflection points were significantly higher in healthy subjects than in patients with CAD for the 24-h and putative awake periods. For the sleep period, the differences were not significant. The percentage of soft words with three inflection points, the most fragmented of this class, tended to be higher in patients with CAD than in healthy subjects. However, significance was only reached for the sleep period.

### 2. Analyses Adjusted for Age and Sex

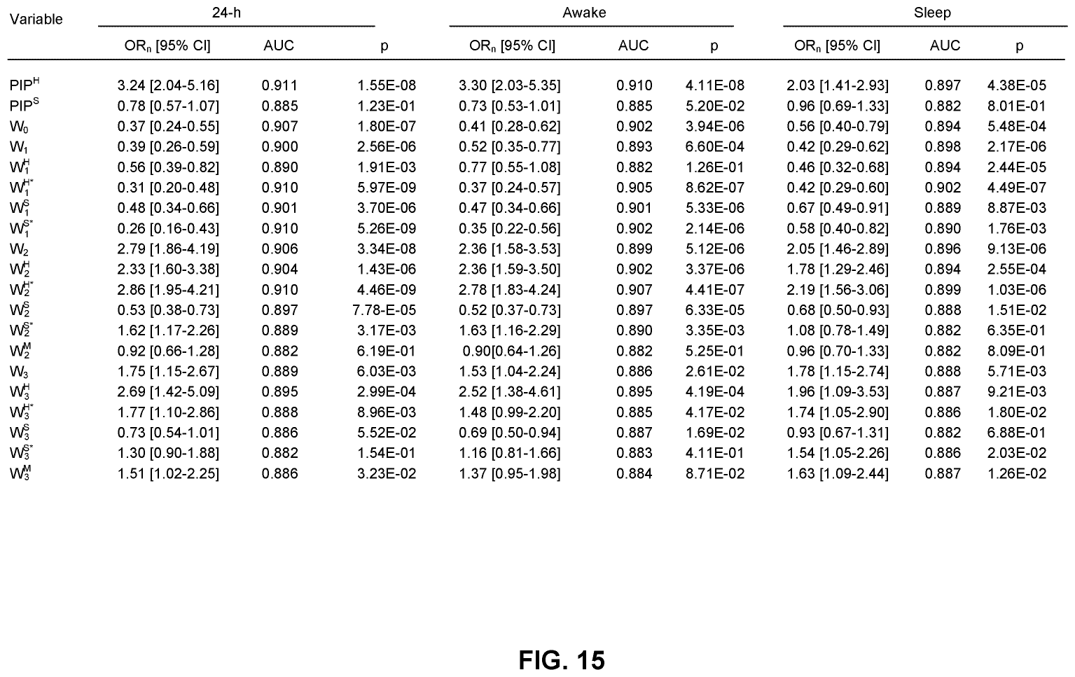

The percentage of hard inflection points, PIP^{H}, remained positively associated with CAD in models adjusted for age and sex (FIG. 15) for the three time periods. The odds of CAD more than tripled, for each one-standard deviation increase in PIP^{H} during the 24-h and putative awake periods. For the putative sleep period, the odds doubled. The percentage of soft inflection points, PIP^{S} tended to be higher in healthy subjects than in those with CAD but the difference did not reach statistical significance for any of the time periods. Adding PIP^{H} to a model with only age and sex significantly improved its performance, whereas PIP^{S} did not.

After adjusting for age and sex, the proportion of fragmented words, W₂ and W₃, remained positively associated with CAD (FIG. 15) for all time periods, while the proportion of fluent words, W₀ and W₁, remained negatively associated with CAD (FIG. 15) for all periods. Specifically, for the 24-h period, a one-standard deviation increase in W₂ and W₃ was associated with an increase of 180 and 75% in the odds of CAD, respectively. In addition, a one-standard deviation increase in W₀ and W₁ was associated with a drop of 63 and 61% in the odds of CAD, respectively. All word groups W₁, 0<j<3, significantly improved the performance of a model with age and sex alone.

Hard word subgroups, Wⱼ^{H} and Wⱼ^{H}* changed with disease in the same way as the groups Wⱼ, (1<j<3). Specifically, W₁^{H} and W₁^{H}* were lower in those with CAD than in healthy subjects, for all time periods. All comparisons were statistically significant except the one with the variable WH1 for the putative awake period. In addition, W₂^{H}, W₃^{H}, W₂^{H}* and W₃^{H}* were significantly higher in those with CAD than in healthy subjects, for all time periods.

Soft word subgroups with one inflection point, W₁^{S} and W₁^{S}* changed with disease in the same way as the fluent, hard word subgroups. Specifically, they were more frequent in healthy subjects than in patients with CAD. In contrast, the percentages of soft words with two and three inflection points were lower in patients with CAD than healthy subjects. The comparison with W₂^{S} were significant for all time periods. For W₃^{S}, only the comparison for the putative awake period reached significance. Mixed words with three inflection points were more discriminatory than those with two.

For the majority of cases (44 out of 54), adding a word group or subgroup to a model with age and sex significantly improved its performance. The exceptions were: W₂^{M}, W₃^{S}, and W₃^{S}* for the 24-h period, W₁^{H}, W₂^{M}, W₃^{S}* and W₃^{M} for the putative awake time, and W₂^{S}*, W₂^{M}, and W₃^{S} for the putative sleep time.

### 3. Analyses Adjusted for Age, Sex and AVNN

The major difference between the results of the analyses adjusted for age and sex and those adjusted for age, sex and AVNN, concerned the variable PIP^{S}. When AVNN was added to the models, the differences in PIP^{S} between patients with CAD and healthy subjects became strongly significant: a one-standard deviation increase in PIP^{S} was associated with an increase in the odds of CAD ranging from 100 to 200%, for the different time periods.

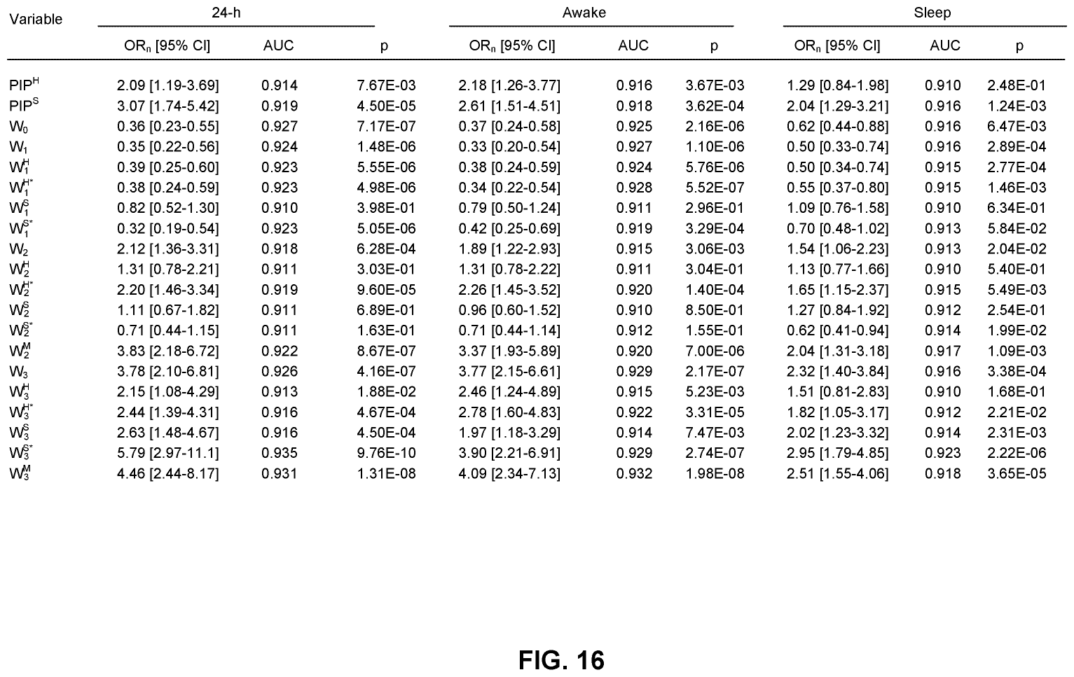

In these models, PIP^{H} remained positively associated with CAD (FIG. 16) for the 24-h and putative awake periods, despite a decrease in the values of the odds ratio. However, for the sleep period, statistical significance was lost.

For all of the time periods, fully adjusted models with word groups without inflection points, with one and three inflection points were more discriminatory than those with two inflection points (FIG. 16). Specifically, a one-standard deviation increase in the percentage of fluent words W₀ and W₁ was associated with a decrease in the odds of CAD ranging from 38 to 64% and from 50 to 67%, respectively. A one-standard deviation increase in the percentage of fragmented words W₃ was associated with an increase in the odds of CAD ranging from 132 to 278%.

Of note, in these fully adjusted models, the number of inflection points, not their type (H, S or M), i.e., the degree of fragmentation of the words, determined the directionality of the effects in the odds of CAD. The majority of the word subgroups significantly improved the performance of a null model with age, sex and AVNN. Within group 1, the most discriminatory word subgroups were W₁^{H} and W₁^{H}*. They appeared in significantly higher densities in healthy subjects than in patients with CAD, for all time periods. Within group 2, W₂^{H}*, and W₂^{M}, were the most discriminatory variables.

For all time periods, significantly higher percentages of these words were observed in patients with CAD than in healthy subjects. Within group 3, all word subgroups were highly discriminatory of the two sample population. The only exception was W₃^{H} during the putative sleep period. As expected, CAD was associated with a significant increase in the density of these words.

### Discussion

Recently, a property of short-term HRV termed fragmentation was described, and a set of metrics to quantify this feature was introduced (Costa I 2017). The key marker of heart rate fragmentation is an overall increase in the frequency of changes in heart rate acceleration sign.

The purpose here was to further explore the property of heart rate fragmentation using symbolic dynamical analysis with the same databases previously studied. The findings were consistent with those reported in Costa I 2017, indicating an increase in heart rate fragmentation with the participants' age and with the presence of CAD. In addition, the symbolic analyses suggested a potentially important dynamical difference between aging in the healthy population and in those with coronary disease. This difference was not anticipated prior to this analysis.

Briefly, the notable findings of the cross-sectional study of heart rate fragmentation with the participants' age were that: (i) in the cohort of healthy subjects, the percentage of soft but not of hard inflection points significantly increased as a function of age; (ii) the percentages of both soft and hard inflection points significantly increased with the participants' age in the cohorts of subjects with CAD; (iii) overall, the density of fluent words tended to decrease and the density of the fragmented ones tended to increase with the participants' age in both populations, for all time periods (FIGS. 12A-12C); (iv) the percentage of words with three hard inflection points, the most fragmented, changed with age differently in the cohorts of healthy subjects and patients with CAD. For the latter, these words markedly increased for all time periods. For the former, the increases did not reach significance. The results from the symbolic analysis adjusted for age, sex and AVNN were consistent with the finding that the overall percentage of transitions from acceleration/deceleration to deceleration/acceleration did not change with the participants' age in the healthy group.

The key findings from the symbolic dynamical analysis comparing healthy subjects and those with CAD were that: (i) the percentages of fluent words, W₀, W₁, W₁^{H}* were significantly higher in healthy subjects than in patients with CAD, for all time periods, in both unadjusted and adjusted models; (ii) the percentages of fragmented words, W₂, W₂^{H}*, W₃, and W₃^{H}*, were significantly lower in healthy subjects than in those with CAD, for all time periods and models; and (iii) although not all word subgroups were statistically different in the two sample populations for all time periods and models, importantly none of the fluent word subgroups, W₁^{H}, W₁^{S}, or W₁^{S}* was significantly higher in patients with CAD than in healthy subjects for any time period or for any model. Similarly, none of the fragmented word subgroups with three inflection points, W₃^{H}, W₃^{H}*, W₃^{M}, W₃^{S}, and W₃^{S}*, was significantly higher in healthy subjects than in patients with CAD for any of the time periods in any model.

Overall, word subgroups with two inflection points were less discriminatory than the other ones, particularly in the fully adjusted models. This finding is not entirely surprising in light of the fact that words with two inflection points encode patterns that represent a transition between dynamical short-term fluency and fragmentation.

Qualitatively similar results to those presented here were obtained from the analysis of RR intervals time series (instead of NN) and words of length 5 (not presented here). Taken together these results robustly support the notion that heart rate fragmentation increases as a function of the participants' age and in the presence of overt CAD.

In this study, words without inflection points included segments of four consecutive accelerative, decelerative and zero acceleration intervals. Excluding the latter, i.e., the segments with no heart rate variability (neither fragmented nor fluent) from the word group W₀, and quantifying their density separately, could potentially allow for a better characterization of a given study population, for example, one with chronic heart failure. In the present study, the results for the word group W₀, including or excluding the word “0000,” were very similar. Therefore, the results for which that word was included were reported. The interpretation of the results for the word group W₀ (with or without the inclusion of the word “0000”) can be dependent on the physiologic context. A deficit of these words is likely a consequence of a high degree of heart rate fragmentation. However, an excess is likely a consequence of long-term (above the normal respiratory frequency) trends in the data. These trends can be pathologic, as seen, for example, with sleep apnea syndromes (see Guilleminault, C., Winkle, R., Connolly, S., Melvin, K., and Tilkian, A. (1984). Cyclical variation of the heart rate in sleep apnea syndrome: mechanisms, and usefulness of 24 h electrocardiography as a screening technique. Lancet 323, 126-131 (herein “Guilleminault 1984)); Lipsitz, L. A., Hashimoto, F., Lubowsky, L. P., Mietus, J., Moody, G. B., Appenzeller, O., et al. (1995). Heart rate and respiratory rhythm dynamics on ascent to high altitude. Heart 74, 390-396 (herein “Lipsitz 1995”); Guzik, P., Piskorski, J., Awan, K., Krauze, T., Fitzpatrick, M., and Baranchuk, A. (2013). Obstructive sleep apnea and heart rate asymmetry microstructure during sleep. Clin. Auton. Res. 23, 91-100 (herein “Guzik 2013”); Jiang, J., Chen, X., Zhang, C., Wang, G., Fang, J., Ma, J., et al. (2017). Heart rate acceleration runs and deceleration runs in patients with obstructive sleep apnea syndrome. Sleep Breath 21, 443-451. (herein “Jiang 2017”)). In other cases, these trends can be physiologic, e.g., when associated with even mild bouts of exercise and recovery. The former conjecture is supported by the work of Guzik 2013, who in a study of heart rate variability in subjects with various degrees of obstructive sleep apnea, found that an increased number of long (>5 intervals) deceleration and acceleration runs were most common in patients with severe sleep apnea.

Symbolic mapping of both the NN interval time series and of its increments have been used in many studies. See Ravelo-Garcia, A. G., Saavedra-Santana, P., Julia-Serda, G., Navarro-Mesa, J. L., Navarro-Esteva, J., Alvarez-Lopez, X., et al. (2014). Symbolic dynamics marker of heart rate variability combined with clinical variables enhance obstructive sleep apnea screening. Chaos 24:024404. (herein Ravelo-Garcia 2014); Cysarz, D., Van Leeuwen, P., Edelhauser, F., Montano, N., Somers, V. K., and Porta, A. (2015). Symbolic transformations of heart rate variability preserve information about cardiac autonomic control. Physiol. Meas. 36, 643-657 (herein “Cysarz 2015”); Yang, A. C., Hseu, S. S., Yien, H. W., Goldberger, A. L., and Peng, C. K. (2003). Linguistic analysis of the human heartbeat using frequency and rank order statistics. Phys. Rev. Lett. 90:108103. (herein “Yang 2003”); Costa, M., Goldberger, A. L., and Peng, C. K. (2005). Broken asymmetry of the human heartbeat: loss of time irreversibility in aging and disease. Phys. Rev. Lett. 95:198102 (herein “Costa 2005”); Costa, M. D., Peng, C. K., and Goldberger, A. L. (2008). Multiscale analysis of heart rate dynamics: entropy and time irreversibility measures. Cardiovasc. Eng. 8, 88-93 (herein “Costa 2008”); Cysarz, D., Bettermann, H., and van Leeuwen, P. (2000). Entropies of short binary sequences in heart period dynamics. Am. J. Physiol. Heart Circ. Physiol. 278, H2163-H2172 (herein “Cysarz 2000”); Cysarz 2015; Kantelhardt, J. W., Ashkenazy, Y., Ivanov, P. C., Bunde, A., Havlin, S., Penzel, T., et al. (2002). Characterization of sleep stages by correlations in the magnitude and sign of heartbeat increments. Phys. Rev. E 65:051908 (herein “Kantelhardt 2002”); Piskorski, J., and Guzik, P. (2011). The structure of heart rate asymmetry: deceleration and acceleration runs. Physiol. Meas. 32, 1011-1023. (“Piskorski 2011”); Guzik, P., Piskorski, J., Barthel, P., Bauer, A., Muller, A., Junk, N., et al. (2012). Heart rate deceleration runs for postinfarction risk prediction. J. Electrocardiol. 45, 70-76 (herein “Guzik 2012”); Guzik 2013; Jiang 2017.

For example, Ashkenazy et al. (2001) used a binary map of the increment time series to analyze the correlation properties of the sign and magnitude heart rate time series of healthy subjects and patients with heart failure. See Ashkenazy, Y., Ivanov, P. C., Havlin, S., Peng, C. K., Goldberger, A. L., Stanley, H. E., et al. (2001). Magnitude and sign correlations in heartbeat fluctuations. Phys. Rev. Lett. 86, 1900-1903. For the shortest time scale explored, 6-16 NN intervals, they found that the dynamics of the sign time series of healthy subjects were closer to brown noise than those of patients with heart failure. This finding supports the hypothesis that long (>5 intervals) deceleration and acceleration runs are more common in healthy subjects than in patients with heart failure.

Guzik et al. (2012, 2013) and Piskorski and Guzik (2011) specifically analyzed the percentages of acceleration and deceleration runs of various lengths in a population of post-infarction patients. Overall, they found that decelerations runs of 2-10 intervals were significantly less frequent in non-survivors and used the runs of lengths 2, 4, and 8 to stratify all-cause mortality risk. The frequency of occurrence of runs of different lengths can be related to the concept of fragmentation. A higher percentage of short (<3) and a lower percentage of longer runs are expected in more fragmented than less fragmented time series. However, there is no direct correspondence between runs of a given length and a specific word. For example, runs of length 3 are necessarily part of the word group W₁, but this word group also includes runs of lengths 1 and 2. Runs of length 2 are part of word groups W₁ and W₂; and runs of length 1 are part of all word groups but W₁.

Cysarz et al. (2000, 2015) and Porta et al. (2007) used a binary map of the increment time series (“1” if ΔRRᵢ₊₁>RRi; “0” if ΔRRᵢ₊₁≤RRᵢ) to analyze putative sympathetic/parasympathetic changes in neuroautonomic control under different conditions. See Porta, A., Tobaldini, E., Guzzetti, S., Furlan, R., Montano, N., and Gnecchi-Ruscone, T. (2007). Assessment of cardiac autonomic modulation during graded head-up tilt by symbolic analysis of heart rate variability. Am. J. Physiol. Heart Circ. Physiol. 293. However, for the types of fragmentation analyses proposed here, binary maps of heart rate increments are not recommended. In fact, if positive (or negative) and zero increments are mapped to the same symbol, soft inflection points are either “ignored” (when an accelerative interval is preceded or followed by an interval in which heart rate does not change), or “transformed” into hard inflection points (when a decelerative interval is preceded or followed by an interval in which heart rate does not change). Consequently, the word groups will contain words with different numbers of inflection points. For example, with the ternary map used, the word group W0 contained only three words, specifically those labeled 0, 80, and 40 in FIG. 10. However, with the binary map, this word group would also include the words 2, 8, 26, 54, 72, and 78 from W₁^{S} (FIG. 10, the words 6, 18, 28, 56, 62, and 74 from subgroup W₂^{S} and the words 20 and 60, from subgroup W₃^{S}, with one, two and three soft inflection points, respectively. The same would be true for other word groups. Thus, the binary mapping of the NN interval time series does not preserve all the information necessary for assessing heart rate fragmentation.

The analyses were based on the definition of acceleration/deceleration as a decrease (increase) in consecutive NN (RR) intervals of <(>) 5 ms. In some cases, any multiple of 5 ms may have been used, but not a lower value, since, as mentioned in the Methods section, the ECG signals were recorded at 200 Hz. ECG signals recorded with a higher sampling frequency (SF) would permit other choices, specifically, any multiple of 1/SF. However, higher resolution/lower thresholds may not necessarily translate into an enhanced ability to discriminate different populations. In fact, the lower the threshold the more likely the results are to be affected by both biological and instrumental noise. On the other hand, the larger the threshold the higher the number of significant changes in acceleration/deceleration that will not be detected. Future studies will help determine an “optimal” range of thresholds for fragmentation analysis.

As noted, increased fragmentation under free-running conditions is not directly attributable to variations in sympathetic or parasympathetic activity (Costa I 2017). These autonomic effectors do not operate on fast enough time scales to account for sustained beat-to-beat changes in heart rate acceleration sign. However, such rapid heart rate acceleration changes have been noted with a variety of pathophysiologic alterations, including subtle premature supraventricular extrasystoles coming from the SA node itself (or from nearby areas), SA exit block variants, modulated sinus parasystole, and possibly mechanical atrial stretch effects (See Friedman 1956; Nazir, S. A., and Lab, M. J. (1996). Mechanoelectric feedback in the atrium of the isolated guinea-pig heart. Cardiovasc. Res. 32, 112-119. (herein “Nazir 1996”). These conditions are most likely to occur with the breakdown of the sinus regulatory control, possibly due to inflammation or fibrosis at various anatomic sites (Ghiassian 2016). In fact, the most common clinical settings of atrial fibrillation, which may represent an end stage of supraventricular fragmentation, are seen with aging and chronic heart disease, conditions in which vagal tone is usually diminished, SA node size is reduced and intercellular coupling may be impaired. See Moghtadaei, M., Jansen, H. J., Mackasey, M., Rafferty, S. A., Bogachev, O., Sapp, J. L., et al. (2016). The impacts of age and frailty on heart rate and sinoatrial node function. J. Physiol. (Lond.) 594, 7105-7126 (herein “Moghtadaei 2016”).

A notable but unanticipated finding of this study was the difference in the pattern of fragmentation seen in the cross sectional analysis of older healthy subjects vs. those with organic heart disease (advanced atherosclerosis). Fragmentation in older healthy subjects was mostly due to the increase in the percentage of transitions from acceleration/deceleration to zero acceleration or vice versa (soft inflection points). Fragmentation in those with CAD, fragmentation was also due to the increase in the percentage of transitions from acceleration to deceleration or vice versa (hard inflection points).

Speculatively, the increase in hard inflection points with disease, i.e., the emergence of beat-to-beat reversals in heart rate acceleration, might also relate to higher degrees of fibrosis and inflammation, substrates for the development of conduction and/or pacemaker abnormalities. The increase in soft inflection points likely relate, in part, to the well-documented decrease in the variance of the NN interval high-frequency fluctuations with aging (Pikkujamsa 1999). In fact, if the structure of the variability is sufficiently preserved (a sign of health), a decrease in the amplitude of the time series, would translate into an increase in the likelihood of having consecutive NN intervals with the same value, that is, of zero accelerations and thus of soft inflection points (assuming that the temporal resolution of the time series does not change).

Although a benign increase in heart rate fragmentation should be rare, it might arise with vagally induced prominent sinus bradycardia with SA Wenckebach, a condition sometimes seen in very healthy (athletic) young subjects. Future studies in well-characterized, larger databases, with outcome data related to incident atrial fibrillation and advanced sinus node disease, should also help ascertaining the translational value of the symbolic analysis of heart rate fragmentation proposed here and the utility of heart rate fragmentation as a quantifiable descriptor of HRV.

Finally, it may be of interest to explore the utility of the concept of dynamical fragmentation and adapt the symbolic dynamic analysis introduced here to the study of changes in repolarization parameters, such as those described under the heading of T wave alternans.

Ultimately, a symbolic dynamical approach to the analysis of heart rate increment time series in a cross-sectional study of healthy subjects and those with CAD, provides evidence supporting the conjecture that fragmentation increases with age and disease. In addition, the results suggest that fragmentation in ostensibly healthy aging is different from fragmentation in the context of overt disease.

### EXAMPLE 2—HEART RATE FRAGMENTATION AS A NOVEL BIOMARKER OF ADVERSE CARDIOVASCULAR EVENTS: THE MULTI-ETHNIC STUDY OF ATHEROSCLEROSIS

The study in this Example describes a novel noninvasive biomarker of cardiovascular (CV) risk. In healthy adult subjects at rest and during sleep, the highest frequency at which the sino-atrial node (SAN) rate fluctuates varies between ˜0.15-0.40 Hz (FIGS. 21A-21C). These oscillations, referred to as respiratory sinus arrhythmia, are due to vagally-mediated coupling between the SAN and breathing. However, not all fluctuations in heart rate (HR) at or above the respiratory frequency are attributable to vagal tone modulation. Under pathologic conditions, an increased density of reversals in HR acceleration sign, not consistent with short-term parasympathetic control, can be observed (FIGS. 21D-21F). This dynamical biomarker of electrophysiologic instability has recently been identified and termed heart rate fragmentation (HRF) (Costa I 2017). In concert, a set of metrics (computational probes) for its quantification was introduced (Costa I 2017; M. D. Costa, R. B. Davis, and A. L. Goldberger. Heart rate fragmentation: a symbolic dynamical approach. Front. Physiol., 8(827):1-14, 2017 (hererin “Costa II 2017”)).

Perhaps the most explicit example of HRF is the subtle supraventricular arrhythmia termed sinus node alternans (Binkley 1995, in which the time between consecutive sinus beats oscillates between two values, short (S) and long (L) following an SLSL pattern. However, HRF includes not only pure (2:1) sinus node alternans but also quasi-periodic and more irregular variants of normal-to normal (NN) alternation. As FIGS. 21A-21F illustrate, clinical recognition of such patterns is difficult from standard electrocardiograms (ECGs). The basic mechanisms of fragmentation, involving either anomalous sinus beats (Lewis 1920) or supraventricular ones originating near the SAN, are unresolved (Costa I 2017, Costa II 2017)

The potential importance of HRF is several-fold. First, it produces a high degree of short-term variability that may be mistaken as a marker of healthy vagal control when standard measures of short-term heart rate variability (HRV) are used. Second, its presence supports the delineation of a new class of biomarkers of cardiac risk. The latter is premised on the conjectured link between HRF and the breakdown in one or more components of the control system (and/or in their interactions) regulating SAN function. Notably, earlier reports of what were first termed “sinus extrasystoles” as well as sinus alternans (Binkely 1995, Lewis 1920) were from patients who were older or who had organic heart disease. Third, the investigation of fragmentation may yield new insights into SAN functionality in health, aging and disease.

Recent studies (Costa I 2017, Costa II 2017), analyzed annotated Holter recordings (University of Rochester Telemetric Holter ECG Warehouse [THEW]) from healthy subjects and patients with advanced coronary artery disease (CAD) using the newly devised HRF metrics. Fragmentation was found to significantly increase as a function of the participants' age in both the healthy population and those with CAD. In contrast, most short-term HRV indices did not significantly change with the participants' age in the CAD group. Furthermore, fragmentation was higher in patients with CAD than in healthy subjects, during both estimated awake and sleep periods, while traditional HRV metrics did not discriminate the two groups.

The general motivation for the present study was to assess the potential utility of the novel indices of HRF as predictors of adverse cardiovascular events (CVEs) and CV mortality, using the large Multi-Ethnic Study of Atherosclerosis (MESA). This ongoing prospective cohort study was designed to investigate the prevalence, correlates and progression of subclinical cardiovascular disease (CVD) in a multi-ethnic population free of overt clinical CVD at study entry. See D. E. Bild, D. A. Bluemke, G. L. Burke, R. Detrano, A. V. Diez Roux, A. R. Folsom, P. Greenland, D. R. Jacob, R. Kronmal, K. Liu, J. C. Nelson, D. O'Leary, M. F. Saad, S. Shea, M. Szklo, and R. P. Tracy. Multi-Ethnic Study of Atherosclerosis: objectives and design. Am. J. Epidemiol., 156(9):871-881, 2002 (herein “Bild 2002”). It was hypothesized that HRF would: 1) be positively associated with cross-sectional age; 2) be positively associated with incident CVEs and CV death; and 3) outperform traditional HR dynamics measures. The study also sought to determine if fragmentation metrics added value to prediction tools computed from static measures, namely the Framingham Heart Study [D'Agostino 2008] and MESA CV risk indices. See R. L. McClelland et al., “10-Year Coronary Heart Disease Risk Prediction Using Coronary Artery Calcium and Traditional Risk Factors: Derivation in the MESA (Multi-Ethnic Study of Atherosclerosis) With Validation in the HNR (Heinz Nixdorf Recall) Study and the DHS (Dallas Heart Study)”, J. Am. Coll. Cardiol., 66(15):1643-1653, 2015.

### Methods

### Study Population and Data Collection

The MESA study has been described in detail previously (Bild 2002). Briefly, over a period of approximately two-years, starting in July 2000, 6,814 persons between the ages of 45 and 84 years of age without evident clinical CVD were recruited at 6 field centers in the US. Institutional review boards from each study site approved the conduct of this study, and written informed consent was obtained from all participants.

A sleep ancillary study was conducted in conjunction with MESA's fifth examination (2010-2013). The study enrolled 2060 participants who underwent unattended, in-home polysomnography (PSG) following a standardized protocol. See S. Redline et al., “Methods for obtaining and analyzing unattended polysomnography data for a multicenter study,” SHHS Research Group. Sleep, 21(3):759-767, 1998. The data obtained using the 15-channel Compumedics Somte System (Compumedics LTd., Abbottsville, Australia) were scored at the Brigham and Women's Hospital centralized reading center by trained technicians using published guidelines. See S. Redline et al., “The scoring of respiratory events in sleep: reliability and validity,” J. Clin. Sleep. Med., 3(2):169-200, 2007. The apnea-hypopnea index (AHI) was calculated based on the average number per hour of sleep of all apneas plus hypopneas associated with ≥3% oxygen desaturation or arousal.

The ECG channels, sampled at 256 Hz, were processed using Compumedics

Somte software for detection and classification of the QRS complexes (R-points) as normal sinus, supraventricular premature or ventricular premature complexes. The automated annotations were reviewed by a trained technician, who made appropriate corrections. Both the NN and the R-to-R (RR) interval time series were analyzed in the present study.

Participants with one or more of the following were excluded: poor signal quality (n=35), pacemaker (n=13), in atrial fibrillation (AF) at the time of the PSG (n=22), <2 hours of combined sleep periods scored as rapid eye movement (REM), stage 1, 2, 3 or 4 (n=16), <75% normal sinus beats between sleep onset and termination (n=11) or no outcome information available after the PSG study (n=11). Participants with CVEs before the PSG (n=185) were excluded from the analyses of the associations between HRV metrics and incident CVEs. These participants were included in analyses of CV mortality.

### Clinical Follow-Up and Event Classification

In addition to clinical exams, participants are followed every 9-12 months to inquire about hospital admissions, CV outpatient diagnoses and procedures, and deaths. Discharge diagnosis codes are obtained for all hospitalizations and medical records are obtained when heart failure, myocardial infarction, stroke, or death are reported. For those over age 65 and enrolled in fee-for-service Medicare, claims data are also used to identify diagnosis and procedure codes. Trained personnel abstracted any hospital records suggesting possible CVEs, which are then adjudicated by physicians at the coordinating center. Nonfatal endpoints in MESA include congestive heart failure, angina, myocardial infarction, percutaneous coronary intervention, coronary bypass grafting or other revascularization procedure, resuscitated cardiac arrest, peripheral arterial disease, stroke (non-hemorrhagic) and transient ischemia attack (TIA). Cardiovascular deaths, as adjudicated committee review, included fatalities directly related to stroke or coronary heart disease. For other deaths, the underlying cause was obtained through state or vital statistics departments. The definition and adjudication of these events have been described in detail previously (Bild, 2002; D. A. Bluemke et al., “The relationship of left ventricular mass and geometry to incident cardiovascular events: the MESA (MultiEthnic Study of Atherosclerosis) study,” J. Am. Coll. Cardiol., 52(25):2148-2155, 2008; J. Yeboah et al., “Comparison of novel risk markers for improvement in cardiovascular risk assessment in intermediate-risk individuals,” JAMA, 308(8):788-795, 2012). The cut-off date for the surveillance period was Dec. 31, 2014.

### Fragmentation Analysis

Fragmentation analysis was performed for 1963 subjects using both NN and RR interval time series. Fragmentation analysis is described in detail in Costa I 2017, Costa II 2017. Briefly, original interbeat interval time series, {sᵢ}, 1≤i≤L (L, time series length) were mapped to a ternary symbolic sequence as follows: “−1” if ΔNNᵢ<−4 ms, “0” if −4<ΔNNᵢ<4 ms, and “1” if ΔNNᵢ>4 ms (Costa II 2017). Note that, since the ECG signals were sampled at 256 Hz, the resolution (τ) of the interbeat interval time series is 1/256˜4 ms. Therefore, only NN (or RR) intervals whose difference was >4 ms or <−4 ms were considered different from each other.

Transitions from HR acceleration to HR deceleration (“−1” to “1”) or vice-versa (“1” to “−1”), and from HR acceleration or HR deceleration to no change in HR (“−1” to “0,” “1” to “0,”) or vice-versa (“0” to “−1,” “0” to “1”) were termed “inflection points.” The percentage of inflection points (PIP) (Costa I 2017) constitutes a measure of HRF reflecting its overall degree of prevalence.

To assess the prevalence of dynamical patterns with increasing degree of fragmentation, the percentages of sequences of 4 consecutive symbols, wi=(s₁, sᵢ₊₁, . . . , s₁₊₁₋₁), 1<i≤L−1+1, termed “words,” with 0, 1, 2 and 3 inflection points were calculated.

These word classes are referred to as W₀, W₁, W₂ and W₃, respectively. The full lexicon that comprises 81 different words is given in (Costa II 2017). Words derived from the NN (RR) interval time series were termed NN (RR) words. The words in groups W₀ and W₁ are the least fragmented (most “fluent”), those in groups W₂ and W₃ are the most fragmented.

### Traditional HRV Analysis

The following traditional time domain HRV indices (See J. Yeboah et al., “Comparison of novel risk markers for improvement in cardiovascular risk assessment in intermediate-risk individuals,” JAMA, 308(8):788-795, 2012) were calculated for 1963 subjects using NN interval time series: 1) the average of all NN intervals (AVNN), 2) mean of the standard deviations of NN intervals in all 5-minute segments (SDNNIDX), 3) the square root of the mean of the squares of differences between adjacent NN intervals (rMSSD) and 4) the percentage of differences between adjacent NN intervals that are greater than 50 ms (pNN50). The following traditional frequency domain HRV indices were calculated: 1) the total spectral power of all NN intervals between 0.15 and 0.4 Hz (HF) and 2) the ratio of low to high frequency power (LF/HF). Each of these metrics was calculated using a 5-minute sliding window (without overlap), with more than 150 beats and more than 75% NN intervals, between sleep onset and sleep termination. A total of 170,527 windows were analyzed. For each subject, the values from the different windows were averaged.

### Statistical Analysis

Continuous variables are summarized as mean±SD. Categorical variables are presented as numbers and percentages.

The associations between independent variables and both incident CVEs and CV mortality were assessed using Cox proportional hazard analysis. Efron's method (See B. Efron, “The efficiency of Cox's likelihood function for censored data,” J. Am. Stat. Assoc., 72(359):557565, 1977) was used to handle ties. Failure time in the individuals with incident CVEs was the time between the PSG study and the time of the diagnosis. For participants without CVEs, the failure time was the time between the PSG study and the latest followup, death, or loss to follow-up. Statistical significance was set at a p-value<0.05. The independent variables were: the fragmentation indices: PIP, W₀, W₁, W₂ and W₃, derived from both NN and RR interval time series; the traditional HRV indices: AVNN, SDNNIDX, rMSSD, pNN50, HF and HF/LF.

Both unadjusted (Model 1) and adjusted models were considered. Adjustments included: i) traditional CV risk factors: age, sex, systolic blood pressure, total cholesterol, high density lipoprotein (HDL) cholesterol, current smoking status, hypertension medication, diabetes and lipid lowering medication (Model 2); ii) the Framingham Heart Study 10-year risk index (Model 3) and iii) the MESA CV risk index without coronary calcification (Model 4).

Standardized hazard ratios (per one-standard deviation increase in the independent variable) were calculated with associated 95% confidence intervals (CI). The assumption of proportional hazards was tested using a global test based on Schoenfeld residuals (See P. M. Grambsch et al., “Proportional hazards tests and diagnostics based on weighted residuals. Biometrika,” 81:515-526, 1994). No violations were noted. The predictive power of the survival models was assessed using Harrell's C statistic. The likelihood ratio test was used to compare the fit of two nested models (null model vs. null model+HRV metric). The three null models considered were those with: i) traditional risk factors (age, gender, systolic blood pressure, total cholesterol, HDL cholesterol, current smoking status, hypertension medication, diabetes and lipid lowering medication), ii) the Framingham, and iii) MESA risk indices. The null hypothesis for each of the likelihood ratio tests was that the two nested models considered fitted the data equally well. Rejection of the null hypothesis implied that the larger model fitted the data better, indicating that a given HRV metric added value to the null model.

Linear regression models with a quadratic term were used to describe the possible nonlinear (U-shaped) relationship between: i) HRF and short-term HRV indices (example of a model with PIP and rMSSD, PIP=β1*ln(rMSSD)+β2*[ln(rMSSD)]²+α, where α is a constant); and ii) short-term HRV indices and the participants' age.

In all analyses, the variables W₀, rMSSD, pNN50, or HF were logarithmically transformed since the models with these log-transformed variables fitted the data better than those with untransformed ones. In all the other cases, the analyses were performed using untransformed independent variables.

### Results

Over a 1086 day average (±231) follow-up period (from the PSG study), 72 participants out of 1771 suffered their first adverse CVE after the PSG study: myocardial infarction (n=16), resuscitated cardiac arrest (n=1), angina (n=14), percutaneous coronary intervention (n=21), coronary bypass graft (n=3), other revascularization (n=6), congestive heart failure (n=10), peripheral vascular disease (n=8), transient ischemic attack (n=5), CV death (n=14) or stroke (non-hemorrhagic brain infarction, n=17). From a total of 1963 participants (1771 without and 192 with prevalent CVEs), 21 died of CVD.

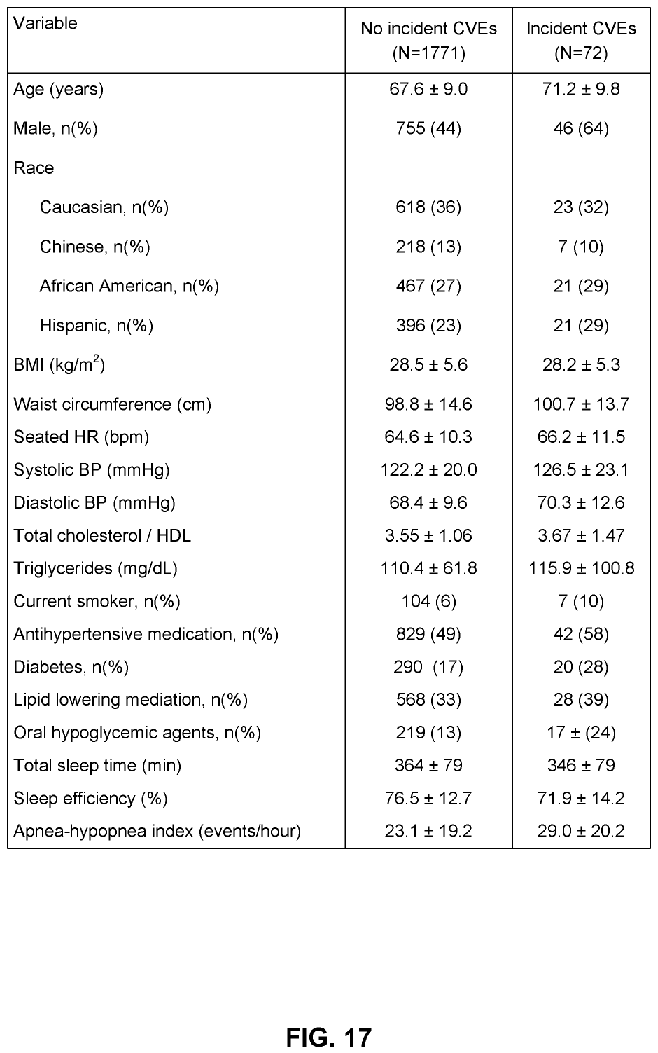

Characteristics of MESA participants without and with a CVE during follow-up are summarized in FIG. 17. Individuals who developed CVEs were older and more likely to be male and have diabetes. They tended to have higher seated HR and higher systolic blood pressure. In addition, this risk group tended to have lower sleep efficiency and a higher apnea-hypopnea index. The differences between those who died and did not were qualitatively similar to the differences between those with and without incident CVEs.

### Relationship of HRF and Traditional HRV Indices with the Participants' Age

All fragmentation indices, derived from either NN or RR interval time series, were significantly associated with the participants' age. The Pearson correlation coefficients (p) for PIP, W₀, W₁, W₂ and W₃ were 0.35, −0.17, −0.31, 0.10 and 0.35, respectively. PIP (FIGS. 24A-24B) and the percentages of fragmented words W₂ and W₃ increased with the participants' age at the estimated rates of 0.28%, 0.09% and 0.35% per year of age, respectively. The percentages of fluent words W₀ and W₁ decreased with the participants' age at the rates of −0.06% and −0.39% per year, respectively. Slightly higher rates of change were observed for the indices derived from RR interval time series (not shown).

The short-term HRV indices, rMSSD, pNN50 and HF, did not vary linearly with the participants' age. Instead, the association between age and short-term variability depended on the participants' age itself. FIGS. 24A-24B illustrate, using the representative example of rMSSD, how the amount of short-term variability varied across different age groups. Variability was higher in both the lowest (<54 yr) and highest (>85 yr) age groups compared to intermediate ones (U-shape relationship). The slope of the relationship between rMSSD and the participants' age increased 1.00 ms per year of age. Above age 66 (vertex of the U-shape relationship), an increase in the participants' age was associated with an increase in rMSSD.

### Unadjusted Analyses of Risk of Incident CVEs

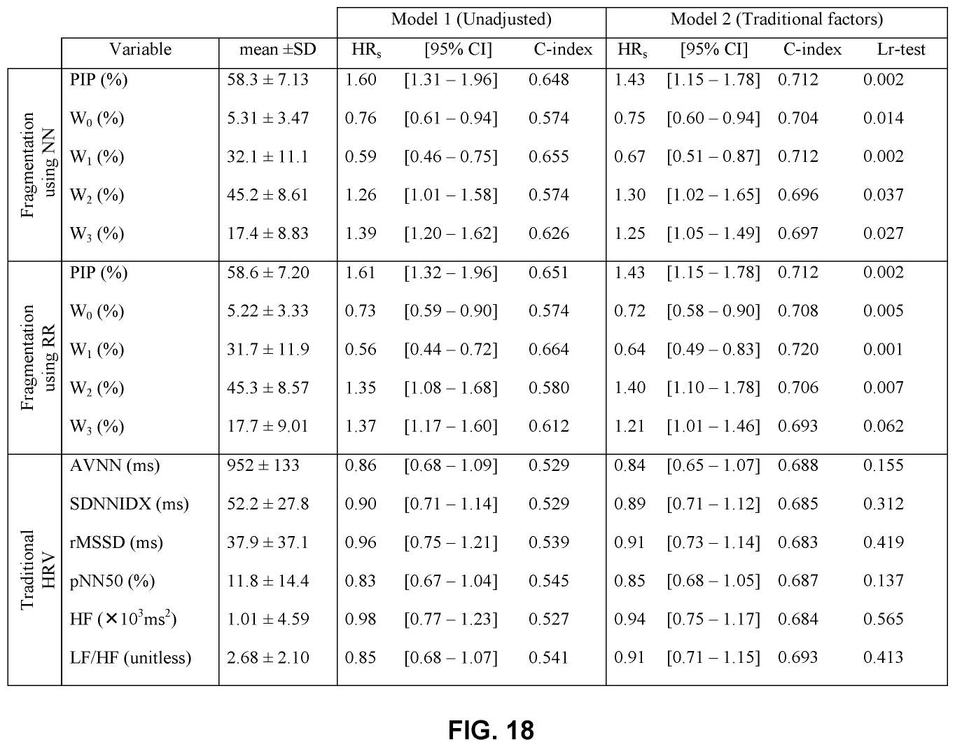

All fragmentation indices, calculated from both the NN and the RR time series, were significantly associated with incident events (FIG. 18; Model 1). The association was positive for fragmented words, W₂ and W₃, as well as PIP, and negative for fluent (less fragmented) words, W₀ and W₁. The most discriminatory of the fragmentation indices was the word W₁ derived from the interval RR time series. A one-standard deviation increase in W₁ was associated with a 44% (95% CI: 28%-66%) decrease in the rate of CVEs. This variable performed comparably to the Framingham Heart Study and MESA CV risk indices (FIG. 22: top panels).

None of the traditional time (AVNN, SDNNIDX, rMSSD, pNN50) and frequency domain (HF, LF/HF) HRV variables were significantly associated with incident events.

### Analyses of Risk of Incident CVEs Adjusted for Traditional Risk Factors

The models with each of the fragmentation and traditional HRV variables were adjusted for standard CV risk factors: age, sex, systolic blood pressure, total cholesterol, HDL cholesterol, current smoking status, hypertension medication, diabetes and lipid lowering medication. In these analyses (FIG. 18: Model 2), all fragmentation indices remained significantly associated with incident CVEs. For example, PIP was associated with a 43% (15%-78%) increased rate of incident CVEs. In addition, all fragmentation indices, with the exception of W₃ calculated from the RR interval time series, added significant value to the model with only the risk factors. In contrast, none of the traditional time (AVNN, SDNNIDX, rMSSD, pNN50) and frequency domain (HF, LF/HF) HRV variables was significantly associated with incident events.

The results did not qualitatively change after adjusting the analyses for each of the following variables: race, body mass index, waist circumference, diastolic blood pressure, use of hypoglycemic agents, total sleep time, sleep efficiency and the apneahypopnea index.

### Analyses of Risk of Incident CVEs Adjusted for the Framingham and MESA CV Risk Indices

In general, the fragmentation indices remained significantly associated with the risk of CVEs in models adjusted for the Framingham and the MESA CV risk indices (FIG. 19). Specifically, increased fragmentation, that is, higher PIP, lower percentages of fluent words W₀ and W₁, and higher percentages of fragmented words W₂ and W₃, were significantly associated with increased risk of events. None of the traditional HRV measures showed any significant association with incident CVEs.

The risk indices in each of these models were also significantly associated with incident CVEs. Specifically, one-standard deviation increase in the Framingham and in the MESA risk indices, was associated with 80% (95% CI: 43%-125%), and 55% (33%-81%) increase in the hazard of adverse CVEs, respectively. Harrell's C statistic was 0.666 and 0.678 for the Framingham and MESA risk indices, respectively. Overall the best model, with a Harrell's C statistic of 0.703, was the one that combined the word group W₁ derived from RR intervals, with the MESA risk index.

Models incorporating the Framingham risk index and any of the fragmentation measures, except W₂ derived from the NN interval time series, performed significantly better than the Framingham index itself. Similarly, all models that included any of the fragmentation measures in addition to the MESA risk index performed significantly better than the MESA index itself.

None of the traditional HRV variables were significantly associated with risk of incident CVEs either in unadjusted or adjusted models. Adding a traditional short-term HRV index to models with fragmentation did not improve their performance.

### Analyses of Risk of CV Death: Unadjusted and Adjusted for the Framingham and MESA Risk Indices

Higher PIP and lower percentage of W₁ words were significantly associated with increased risk of CV death in unadjusted analyses as well as in analyses adjusted for the Framingham and the MESA CV risk indices (FIG. 20 and FIG. 22). Specifically, a one-standard deviation (˜7%) increase in PIP derived from the analysis of NN interval time series was associated with an increase in the rate of CV death of 89% in unadjusted models and of 67% and 65% in models adjusted for the Framingham and the MESA risk indices, respectively. A one-standard deviation (˜11%) increase in the percentage of W₁ (“fluent” or least fragmented) words, also derived from the analysis of NN interval time series, was associated with a decrease in the rate of CV death of 59% in unadjusted models, and of 52% and 55% in models adjusted for Framingham and the MESA risk indices, respectively. Similar results were obtained from the analyses of the RR interval time series. Further adjusting the models by prevalent CVD did not change the significance of the associations between the fragmentation metrics and risk of CV death.

Lower percentages of fluent words W₀ were associated with increased risk of CV death in all models. However, these associations were weaker than those with word W₁. The percentages of word W₂ and W₃, the most fragmented, were positively associated with the risk of CV death in all models. However, the significance of the associations depended on the particular model (FIG. 20).

One-standard deviation increase in the Framingham (˜9) and in the MESA risk (˜6) indices was associated with 137% (95% CI: 48%-278%) and 62% (35%-96%) increase in the rate of CV death, respectively. Harrell's C statistic for the former and latter models was 0.749 and 0.797, respectively. Both variables, PIP and W₁, calculated from NN and RR interval time series, added significant information to models with either of these risk indices. The results for word groups W₀, W₂ and W₃ depended on the particular model. Overall, the best model, with a Harrell's C statistic of 0.838, was the one that combined the word W₁, derived from RR intervals, with the MESA risk index.

None of the traditional HRV variables were significantly associated with risk of CV death either in unadjusted or adjusted models. Adding a traditional short-term HRV index to models with fragmentation did not improve their performance.

### Relationship between HRF and Short-Term HRV Indices

Nonlinear (U-shape) relationships were found between fragmentation indices and measures of short-term variability. FIG. 23 shows one representative example, the relationship between PIP and ln(rMSSD). For the first three quartiles of rMSSD values, (i.e., for rMSSD values below the 75th percentile of rMSSD, specifically, ln(rMSSD)<3.7 ms), the degree of fragmentation and the amount of short-term variability were inversely correlated. In the upper quartile of rMSSD values, the degree of fragmentation and the amount of short-term variability were positively associated. Qualitatively similar results were found for pNN50 and HF power.

### Discussion

The present investigation was designed to test the association of quantitative measures of HRF, a newly defined property of short-term sino-atrial rhythm dynamics, with adverse CV outcomes in MESA, a large ongoing multicenter study of individuals recruited from the general community. The key findings of this study were that: 1) increased HRF was significantly associated with risk of incident CVEs and CV mortality; 2) measures of fragmentation added value to Framingham and MESA risk prediction indices; and 3) traditional metrics of short-term HRV were not associated with incident CVEs and CV death.

The development of the concept of fragmentation and its quantitative metrics was motivated in part by an apparent paradox in the results of traditional time and frequency domain analyses of a number of studies (See Costa I 2017; D. Raman et al., “Polysomnographic heart rate variability indices and atrial ectopy associated with incident atrial fibrillation risk in older community-dwelling men,” JACC. Clin. Electrophysiol., 3(5):451-460, 2017; P. K. Stein et al., “Sometimes higher heart rate variability is not better heart rate variability: results of graphical and nonlinear analyses. J. Cardiovasc. Electrophysiol.,” 16(9):954-959, 2005; P. E. Drawz et al., “Heart rate variability is a predictor of mortality in chronic kidney disease: a report from the CRIC Study.” Am. J. Nephrol., 38(6):517-528, 2013; M. C. de Bruyne et al., “Both decreased and increased heart rate variability on the standard 10-second electrocardiogram predict cardiac mortality in the elderly: the Rotterdam Study,” Am. J. Epidemiol., 150(12):1282-1288, 1999; H. V. Huikuri et al, “Clinical application of heart rate variability after acute myocardial infarction,” Front. Physiol., 3:41, 2012). While the mechanisms of the relatively slow (i.e., below the respiratory frequency) variations in HR are attributable to complex interactions between the parasympathetic and the sympathetic effects of the autonomic nervous system, faster variations in the range of 0.15-0.40 Hz are mainly attributed to vagal tone modulation (HRV 1996). Therefore, short-term (high frequency) measures of HR dynamics, such as rMSSD, pNN50 and HF power, are typically interpreted as surrogate measures of cardiac vagal tone. In contexts where cardiac vagal tone modulation is known to be diminished, for example, with advanced aging and established CVD, these “vagal” measures are expected to be lower. In fact, a monotonic decrease in high frequency variability with increasing age is generally observed in cross-sectional studies of ostensibly healthy adults (See W. T. O'Neal et al., “Reference ranges for short-term heart rate variability measures in individuals free of cardiovascular disease: The Multi-Ethnic Study of Atherosclerosis (MESA),” J. Electrocardiol., 49(5):686-690, 2016). Furthermore, extremely low variability has been consistently reported as associated with adverse outcomes (HRV 1996; Huikuri 2012; S. M. Pikkujamsa et al., “Cardiac interbeat interval dynamics from childhood to senescence,” Circulation, 100(4):393-399, 1999).

However, in certain cases just the opposite has been reported (Grambsch 1994; de Bruyne 1999; A. Goette et al., “EHRA/HRS/APHRS/SOLAECE expert consensus on atrial cardiomyopathies: Definition, characterization, and clinical implication,” Heart Rhythm, 14(1):e3-e40, 2017); namely a paradoxical increase in short-term HR fluctuations occurring in contexts where reduced vagal tone would have been expected based on age and/or advanced heart disease. In the present study, a U-shaped relationship was observed between traditional short-term HRV measures and cross-sectional age (FIGS. 24A-24B). From approximately ages 45 to 65 years the amount of short-term variability decreased. Subsequently, variability increased despite the well-known decrease in cardiac vagal tone modulation with advancing age. These results provide further evidence that in cohorts of middle-aged to elderly individuals, such as MESA, traditional HRV indices may fail to reflect accurately changes in cardiac vagal tone.

The term “fragmented heart rate” was coined to refer to rhythms in which HR acceleration sign changes at a frequency higher than that attributable to vagal tone modulation of the SAN. These rhythms include but are not limited to classic sinus alternans and its variants. If the amplitude of the fluctuations is low (e.g., ≲80 ms), fragmentation is unlikely to be detected in clinical readings of short (typically 10 seconds) and long (Holter) ECG recordings.

An intuitive measure of fragmentation is the percentage of changes in HR acceleration sign, that is, PIP, in NN (or RR) time series. Most recently, a symbolic dynamical approach was introduced (Costa II 2017) that quantifies the frequency of occurrence of different patterns of fluctuations, from least fragmented (most fluent) to most fragmented. These fragmentation indices were originally tested in studies of publicly available databases from the Rochester THEW archives.

The origins of HRF remain speculative. Possible pathophysiologic mechanisms include increased automaticity in or proximal to the SAN, exit block in the SAN area, modulated sinus/atrial parasystole, or beat-to-beat changes due to perturbations in atrial stretch receptors (Costa I 2017, Costa II 2017). These electrophysiologic perturbations, in turn, may be related to underlying atrial (See A. Goette et al., “EHRA/HRS/APHRS/SOLAECE expert consensus on atrial cardiomyopathies: Definition, characterization, and clinical implication,” Heart Rhythm, 14(1):e3-e40, 2017; M. Sosnowski et al., “Heart rate variability. Is it influenced by disturbed sinoatrial node function?”, J. Electrocardiol., 28(3):245-251, 1995; K. C. Roberts-Thomson et al., “Sinus node disease: an idiopathic right atrial myopathy,” Trends Cardiovasc. Med., 17(6):211-214, 2007) or ventricular disease. Systemic factors that may also contribute to pathophysiologic dysregulation include inflammation, degeneration, fibrosis and calcification (Costa I 2017). Future experimental and mathematical modeling studies will hopefully shed light on the putative links between these and other mechanisms and fragmentation. Possible genomic associations with HRF remain to be explored.

Based on the analyses of the THEW databases (Costa I 2017, Costa II 2017), it was hypothesized that increased HRF might be a biomarker of increased risk of incident CVEs and CV death. To explore these hypotheses, HR dynamics from a subset of the participants in the MESA were analyzed. This national study is one of the largest prospective investigations designed to track meticulously the course of CVD in an ethnically diverse population free of overt clinical CVD at study entry. Two types of ECG recordings with detailed follow-up data were available at the time of this analysis: traditional 10-second ECGs and the ECG channel of the PSG studies. It was chosen to examine the latter given the nonstationary nature of HR dynamics, (which implies that statistical time series analysis tools are most reliable when applied to “long” recordings (HRV 1996)), and the fact that previous studies (Costa I 2017, Costa II 2017) have shown that the discriminatory power of HRF was comparable during awake and sleep periods.

A corollary finding was the absence of a association between the most commonly used traditional short-term HRV measures and incident CVEs in unadjusted and adjusted models. These results are not as surprising as they might appear at first glance. First, the U-shaped relationship between traditional short-term HRV measures and the participants' age (FIGS. 24A-24B) was indicative that such measures would be of limited utility in this cohort. Second, as previously mentioned, traditional measures of HRV, in contrast to fragmentation measures, also failed to discriminate patients with CAD from ostensibly healthy subjects in databases provided by the University of Rochester (Costa I 2017, Costa II 2017). Third, HRF, by increasing variability not ascribable to physiologic vagal tone modulation may confound the results of traditional HRV. The nonlinear (U-shaped) relationship between HRF and short-term variability (FIG. 23) supports this conjecture.

In fact, the subgroups of participants with the lowest and highest amounts of variability, presumed to have the highest and lowest risk of adverse events, respectively, both showed increased HRF. These findings support the reports of Stein and colleagues (see P. K. Stein et al., “Development of more erratic heart rate patterns is associated with mortality post-myocardial infarction,” J. Electrocardiol., 41(2):110-115, 2008; P. K. Stein, “Heart rate variability is confounded by the presence of erratic sinus rhythm,” Comput. Cardiol., 26:669-672, 2002) and others (Drawz 2013; de Bruyne 1999; Huikuri 2012).

Of note, fragmentation and traditional HRV indices differ in the following major way. By construction, fragmentation indices do not mathematically depend on mean HR and/or the amplitude of its fluctuations. These salient attributes derive from the fact that accelerations/decelerations are defined as increments/decrements in HR of any magnitude. In contrast, by definition, short-term HRV indices quantify information that is encoded in the amplitude of the fluctuations. As previously mentioned rMSSD, pNN50 and HF power were not associated with risk of incident CVEs and CV mortality. The other widely used HRV metrics, AVNN, SDNNIDX and LF/HF were also not associated with risk of incident CVEs and CV mortality. Furthermore, none of the traditional indices improved the performance of models that included a fragmentation index.

Of potential basic and translational relevance, both the NN and RR time series were used. The former were employed using expert edited time series from THEW and MESA to insure that the fragmentation was likely related to beats originating in or near to SAN, therefore not distinguishable from sinus beats, at least from the single lead provided. The RR time series were used to demonstrate that fragmentation analysis, not relying on detailed beat annotation, had comparable (or even superior) discriminatory power to that employing NN time series, substantially facilitating the development of automatable analyses.

Finally, it is worth emphasizing that the Framingham and the MESA indices are composite measures incorporating information related to demographics (age, sex, race), lifestyle (smoking status), vital signs (blood pressure) and blood analytes (lipids, glucose). In contrast, HRF is a single index reflecting the frequency of the changes in HR acceleration sign. How can such a single metric based on a continuous ECG keep “pace” with these other multivariable risk stratification tools? The answer may relate in part to the fact that HRF indices are dynamical measures, not static probes. In contrast, blood pressure, cholesterol, glucose and others common biomarkers are “snapshot” readouts. Thus, they provide limited information on the dynamics of the underlying control mechanisms.

More generally, HRF metrics belong to the new class of dynamical probes (A. L. Goldberger, “Giles F. Filley lecture. Complex systems,” Proc Am Thorac Soc, 3(6):467-471, 2006; M. D. Costa et al., “Dynamical glucometry: use of multiscale entropy analysis in diabetes,” Chaos, 24(3):033139, 2014; [28]) that can report, in real-time, on salient aspects of integrative, multiscale, regulatory systems and of their breakdown with aging and disease. The use of these probes may enhance the clinical utility of traditional risk assessment tools (FIG. 19 and FIG. 20) and of other emerging technologies, such as genomic profiling. In furtherance of the goals of precision medicine, the dynamical property of HRF may also constitute a novel target for therapeutic interventions.

### CONCLUSION

Analysis of short-term HRV is enhanced by a set of computational tools that quantify the fragmentation of heartbeat variability, defined by abrupt changes in the sign of HR acceleration. For example, heart rate fragmentation (HRF) is a manifestation of anomalous short-term sino-atrial variability. In a Holter monitor database from healthy subjects, the degree of fragmentation increased with the participants' age. In particular, HRF was associated with increased risk of cardiac adverse events and cardiac mortality in MESA. Furthermore, fragmentation measures outperformed traditional short-term measures of HRV in discriminating a group of patients with CAD and from the healthy subjects. Fragmentation of sinus rhythm cadence may support a new class of dynamical biomarkers that probe the integrity of the regulatory network comprising neuroautonomic, sinus node and atrial components.

FIGS. 1-24 as described herein are illustrative examples allowing an explanation of the present invention. It should be understood that embodiments of the present invention could be implemented in hardware, firmware, software, or a combination thereof. In such an embodiment, the various components and steps would be implemented in hardware, firmware, and/or software to perform the functions of the present invention. That is, the same piece of hardware, firmware, or module of software could perform one or more of the illustrated blocks (i.e., components or steps).

The present invention can be implemented in one or more computer systems capable of carrying out the functionality described herein. Referring to FIG. 25, an example computer system 2500 useful in implementing the present invention is shown. Various embodiments of the invention are described in terms of this example computer system 2500. After reading this description, it will become apparent to one skilled in the relevant art(s) how to implement the invention using other computer systems and/or computer architectures.

The computer system 2500 includes one or more processors, such as processor 2504. The processor 2504 is connected to a communication infrastructure 2506 (e.g., a communications bus, crossover bar, or network).

Computer system 2500 can include a display interface 2502 that forwards graphics, text, and other data from the communication infrastructure 2506 (or from a frame buffer not shown) for display on the display unit 2530.

Computer system 2500 also includes a main memory 2508, preferably random access memory (RAM), and can also include a secondary memory 2510. The secondary memory 2510 can include, for example, a hard disk drive 2512 and/or a removable storage drive 2514, representing a floppy disk drive, a magnetic tape drive, an optical disk drive, etc. The removable storage drive 2514 reads from and/or writes to a removable storage unit 2518 in a well-known manner. Removable storage unit 2518, represents a floppy disk, magnetic tape, optical disk, etc. which is read by and written to removable storage drive 2514. As will be appreciated, the removable storage unit 2518 includes a computer usable storage medium having stored therein computer software (e.g., programs or other instructions) and/or data.

In alternative embodiments, secondary memory 2510 can include other similar means for allowing computer software and/or data to be loaded into computer system 2500. Such means can include, for example, a removable storage unit 2522 and an interface 2520. Examples of such can include a program cartridge and cartridge interface (such as that found in video game devices), a removable memory chip (such as an EPROM, or PROM) and associated socket, and other removable storage units 2522 and interfaces 2520 which allow software and data to be transferred from the removable storage unit 2522 to computer system 2500.

Computer system 2500 can also include a communications interface 2524. Communications interface 2524 allows software and data to be transferred between computer system 2500 and external devices. Examples of communications interface 2524 can include a modem, a network interface (such as an Ethernet card), a communications port, a PCMCIA slot and card, etc. Software and data transferred via communications interface 2524 are in the form of signals 2528 which can be electronic, electromagnetic, optical, or other signals capable of being received by communications interface 2524. These signals 2528 are provided to communications interface 2524 via a communications path (i.e., channel) 2526. Communications path 2526 carries signals 2528 and can be implemented using wire or cable, fiber optics, a phone line, a cellular phone link, an RF link, free-space optics, and/or other communications channels.

In this document, the terms “computer program medium” and “computer usable medium” are used to generally refer to media such as removable storage unit 2518, removable storage unit 2522, a hard disk installed in hard disk drive 2512, and signals 2528. These computer program products are means for providing software to computer system 2500. The invention is directed to such computer program products.

Computer programs (also called computer control logic or computer readable program code) are stored in main memory 2508 and/or secondary memory 2510. Computer programs can also be received via communications interface 2524. Such computer programs, when executed, enable the computer system 2500 to implement the present invention as discussed herein. In particular, the computer programs, when executed, enable the processor 2504 to implement the processes of the present invention described above. Accordingly, such computer programs represent controllers of the computer system 2500.

In an embodiment where the invention is implemented using software, the software can be stored in a computer program product and loaded into computer system 2500 using removable storage drive 2514, hard disk drive 2512, interface 2520, or communications interface 2524. The control logic (software), when executed by the processor 2504, causes the processor 2504 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to one skilled in the relevant art(s).

In yet another embodiment, the invention is implemented using a combination of both hardware and software.

In one example embodiment, the present invention can be implemented in a computer-based monitor unit for use in a clinical setting. In another embodiment, the present invention can be implemented in an ambulatory unit akin to a Holter monitor, personal computing device, or similar portable device. In yet another embodiment, the present invention can be implemented in an implantable medical device such as an implantable cardioverter defibrillator (ICD).

In summary, the approach described herein met the objectives of: 1) introduce a set of metrics designed to probe the degree of sinus rhythm fragmentation; 2) test the hypothesis that the degree of fragmentation of heartbeat time series increases with the participants' age in a group of healthy subjects; 3) test the hypothesis that the heartbeat time series from patients with advanced coronary artery disease (CAD) are more fragmented than those from healthy subjects; and 4) compare the performance of the new fragmentation metrics with standard time and frequency domain measures of short-term HRV. The methods used in the approach described herein included: analysis of annotated, open-access Holter recordings (University of Rochester Holter Warehouse) from healthy subjects and patients with CAD using these newly introduced metrics of heart rate fragmentation, as well as standard time and frequency domain indices of short-term HRV, detrended fluctuation analysis and sample entropy. The results of the approach described herein included the following. The degree of fragmentation of cardiac interbeat interval time series increased significantly as a function of age in the healthy population as well as in patients with CAD. Fragmentation was higher for the patients with CAD than the healthy subjects. Heart rate fragmentation metrics outperformed traditional short-term HRV indices, as well as two widely used nonlinear measures, sample entropy and detrended fluctuation analysis short-term exponent, in distinguishing healthy subjects and patients with CAD. The same level of discrimination was obtained from the analysis of normal-to-normal sinus (NN) and cardiac interbeat interval (RR) time series. Conclusions of the approach described herein included the following. The fragmentation framework and accompanying metrics introduced here constitute a new way of assessing short-term HRV under free-running conditions, one which appears to overcome salient limitations of traditional HRV analysis. Fragmentation of sinus rhythm cadence may provide new dynamical biomarkers for probing the integrity of the neuroautonomic-electrophysiologic network controlling the heartbeat in health and disease.

The foregoing description of the specific embodiments will so fully reveal the general nature of the invention that others can, by applying knowledge within the skill of the art (including the contents of the documents cited and incorporated by reference herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the art.

While various embodiments of the present invention have been described above, it should be understood that they have been presented by way of example, and not limitation. It will be apparent to one skilled in the relevant art(s) that various changes in form and detail can be made therein without departing from the spirit and scope of the invention. Thus, the present invention should not be limited by any of the above-described exemplary embodiments, but should be defined only in accordance with the following claims and their equivalents.

What is claimed is:

1. A method of assessing cardiovascular risk of a subject, comprising:
receiving a first set of electrocardiogram (ECG) signals of the subject;
analyzing data from the first set of ECG signals to identify sign changes in heart rate
acceleration in the first set of ECG signals;
determining a degree of fragmentation in the first set of ECG signals based on the identified sign changes in heart rate acceleration; and
assessing cardiovascular risk of the subject based on the degree of fragmentation.

2. The method of claim 1, wherein analyzing data from the first set of ECG signals further comprises:
deriving, from each ECG signal, a time series of normal-to-normal (NN) intervals, {NNᵢ}={t_{N}_{ᵢ}−t_{N}_{_{i−1}}}, wherein t_{N}_{ᵢ} represents the time of occurrence of the i^{th} normal sinus beat, and the time series of the differences between consecutive NN interval increments, {ΔNNᵢ}={NNᵢ−NNᵢ₋₁}; and
computing a set of fragmentation indices from the time series derived from each ECG signal.

3. The method of claim 2, wherein a fragmentation index in the set of fragmentation indices comprises: a percentage of zero-crossing points in the time series of the NN intervals or a percentage of inflection points (PIP) in the time series of the NN intervals.

4. The method of claim 2, wherein a fragmentation index in the set of fragmentation indices comprises an inverse of an average length of acceleration and deceleration NN segments (IALS_{NN}), wherein the acceleration and deceleration segments are sequences of NN intervals between consecutive inflection points for which the differences between two NN intervals are <0 and >0, respectively, and wherein a length of a segment is the number of NN intervals in the segment.

5. The method of claim 2, wherein a fragmentation index in the set of fragmentation indices comprises: a percentage of short NN segments (PSS_{NN}), wherein PSS_{NN} further comprises a complement of a percentage of NN intervals in acceleration and deceleration segments with three or more NN intervals.

6. The method of claim 2, wherein a fragmentation index in the set of fragmentation indices comprises: a percentage of NN intervals in alternation segments, wherein each alternation segments comprises a sequence of at least four NN intervals, for which heart rate acceleration changes sign every beat.

7. The method of claim 2, further comprising:
applying the set of fragmentation indices to the data from the first set of ECG signals.

8. The method of claim 7, further comprising:
further determining the degree of fragmentation in the first set of ECG signals based on values of the set of fragmentation indices, wherein the degree of fragmentation increases based on an increase in the values of the set of fragmentation indices.

9. The method of claim 1, wherein analyzing data from the first set of ECG signals further comprises:
deriving, from each ECG signal, a time series of cardiac interbeat (RR) intervals, {RRᵢ}={t_{R}_{ᵢ}−t_{R}_{_{i−1}}}, wherein t_{R}_{ᵢ} represents the time of occurrence of the i^{th} QRS complex, and the time series of the differences between consecutive RR intervals (increments), {ΔRRᵢ}={RRᵢ−RRᵢ₋₁}; and
computing a set of fragmentation indices from the time series derived from each ECG signal.

10. The method of claim 9, wherein a fragmentation index in the set of fragmentation indices comprises: a percentage of zero-crossing points in the RR time series or a percentage of inflection points (PIP) in the time series of the RR intervals.

11. The method of claim 9, wherein a fragmentation index in the set of fragmentation indices comprises: an inverse of an average length of acceleration and deceleration RR segments (IALS_{RR}), wherein the acceleration and deceleration segments are sequences of RR intervals between consecutive inflection points for which the differences between two RR intervals are <0 and >0, respectively, and wherein a length of a segment is the number of RR intervals in the segment.

12. The method of claim 9, wherein a fragmentation index in the set of fragmentation indices comprises: a percentage of short RR segments (PSS_{RR}), wherein PSS_{RR} further comprises a complement of a percentage of RR intervals in acceleration and deceleration segments with three or more RR intervals.

13. The method of claim 9, wherein a fragmentation index in the set of fragmentation indices comprises: a percentage of RR intervals in alternation segments, wherein each alternation segments comprises a sequence of at least four RR intervals, for which heart rate acceleration changes sign every beat.

14. The method of claim 9, further comprising:
applying the set of fragmentation indices to the data from the first set of ECG signals.

15. The method of claim 1, further comprising:
mapping the differences between consecutive NN or RR intervals above and below given thresholds in the first set of ECG signals to at least three different symbols;
identifying different segments of consecutive symbols in the plurality of symbols as a plurality of words;
determining a plurality of word groups based on identifying for each word the number and types of transitions between different symbols;
determining percentages of each word group; and
quantifying the degree of fragmentation in the first set of ECG signals based on the percentages of each word group in the plurality of word groups.
